(19) United States
(12) Patent Application Publication
(10) Pub. No.: US 2012/0245460 A1
(43) Pub. Date: Sep. 27, 2012

(54) METHOD OF DETERMINING ISCHEMIA USING PAIRED STRESS AND REST SCANS

(75) Inventors: Piotr J. Slomka, Los Angeles, CA (US); Daniel S. Berman, Los Angeles, CA (US); Guido Germano, Los Angeles, CA (US)

(73) Assignee: CEDARS-SINAI MEDICAL CENTER, Los Angeles, CA (US)

(21) Appl. No.: 13/513,821

(22) PCT Filed: Dec. 3, 2010
(86) PCT No.: PCT/US10/58949

§ 371 Date: Jun. 4, 2012

Related U.S. Application Data
(60) Provisional application No. 61/266,458, filed on Dec. 3, 2009.

(51) Int. Cl. A61B 5/02; A61B 5/0402; A61B 6/00

(52) U.S. Cl. 600/425; 600/508; 600/509

(57) ABSTRACT

A method of identifying perfusion abnormalities in a heart of a patient. The method is performed with a patient stress map including stress values, a patient rest map including rest values, and one or more normal maps. The normal maps may include a normal change limit map including change limits, and a normal stress limit map including stress limits. The stress and rest maps are co-registered with one another and the normal maps. The method includes creating a patient change map by subtracting the rest count values of the rest map from the stress count values of the co-registered stress map. Then, in some embodiments, the patient stress and change maps are jointly compared to the normal stress and change limit maps to detect one or more hypoperfused regions. In such embodiments, the one or more regions detected are identified as having perfusion abnormalities and optionally displayed.

### CROSS REFERENCE TO RELATED APPLICATION(S)

This application claims the benefit of U.S. Provisional Application No. 61/266,458, filed Dec. 3, 2009, which is incorporated herein by reference in its entirety.

### STATEMENT REGARDING FEDERALLY SPONSORED RESEARCH OR DEVELOPMENT

This invention was made with U.S. government support under grant number R01HL089765-01 awarded by the National Heart, Lung, and Blood Institute (“NHLBI”) of the National Institutes of Health (“NIH”). The U.S. Government has certain rights in the invention.

### BACKGROUND OF THE INVENTION

1. Field of the Invention

The present invention is directed generally to methods of detecting and evaluating ischemia in heart muscle.

2. Description of the Related Art

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Ischemia is an inadequate blood supply to an area of the body caused by a blockage in one or more blood vessels that deliver blood to that area. A pair of single-photon emission computerized tomography (“SPECT”) scans that each image the left ventricle of a patient's heart may be used to detect ischemia in heart muscle. The pair of scans includes (1) a stress myocardial perfusion SPECT (“MPS”) scan performed when the patient is under stress and (2) a rest MPS scan performed when the patient is at rest.

The MPS scanner detects an amount of a radioactive marker or tracer (e.g., technetium-99m sestamibi) that was injected into the bloodstream and subsequently perfused into the heart muscle of the left ventricle. The MPS scan captures multiple images of the radioactive tracer from outside the chest. Generally, images are collected at different locations about a 180 degree arc oriented substantially perpendicularly to a longitudinal (or long) axis of the body.

A three-dimensional representation is typically constructed from the longitudinally captured images. Further, “short axis” images, which are images perpendicular to the longitudinal axis of the body, may also be constructed. When the left ventricle is imaged, these short axis images are typically ring-shaped while the longitudinally captured images are generally U-shaped.

The three-dimensional representation may be sampled to create a polar map, which is a two-dimensional representation of the short axis images of the three-dimensional model. The polar map may be displayed on a conventional computer monitor. Thus, the displayed polar map may be described as including pixels. Each pixel may correspond to one or more samples. Alternatively, a sample may be represented by more than one pixel.

Each sample has a count value and a location value corresponding to a location within the left ventricle from which the sample is believed to have been obtained. The count value indicates density of the radioactive tracer at the location value. If a pixel corresponds to a single sample, the pixel may be assigned the count value of the sample. If a pixel corresponds to more than one sample, the pixel may be assigned an aggregated count value determined based on a combination (e.g., an average, a median value, and the like) of the count values of the samples represented by the pixel. If a sample is represented by more than one pixel, each pixel may be assigned the count value of the sample.

Because an ischemic area has an inadequate blood supply, an ischemic area of the heart will have less radioactive tracer perfused therein than a healthy area. Within the polar map, the density of the radioactive tracer within a region may be used to evaluate perfusion in that region. Thus, areas having perfusion abnormalities will typically have lower count values than healthy areas.

The presence of perfusion abnormalities in the stress MPS scan that are smaller or absent in the rest MPS scan indicate ischemia. Hence, assessment of myocardial perfusion at stress and rest may be essential for the diagnosis of coronary artery disease (“CAD”) and risk stratification of patients with ischemic heart disease. See Parisi A. F., Hartigan P. M. and Folland E. D., Evaluation of exercise thalium scintigraphy versus exercise electrocardiography in predicting survival and outcome and morbid cardiac events in patients with single- and double-vessel disease: findings from Angioplasty Compared to Medicine (ACME) Study. J. Am. Coll. Cardiol., 1997; 30: 1256-1263.

Stress data may be obtained from the stress MPS scan and rest data may be obtained from the rest MPS scan. In current quantification protocols, the stress and rest data are fitted separately to a geometric stress polar map and a geometric rest polar map, respectively. In other words, at least one patient stress polar map is created using the stress data and at least one patient rest polar map is created using the rest data.

Normal stress samples may be obtained from stress MPS scans performed on normal or subjects with low likelihood of disease and visually normal scans. These normal stress samples may be used to create normal stress limit values (which may be used to construct a normal stress limit polar map). Further, normal rest samples may be obtained from rest MPS scans performed on the subjects (with low likelihood of disease and visually normal scans) and used to create normal rest limit values (which may be used to construct a normal rest limit polar map). The normal stress and rest limit values are typically stored in one or more databases. For example, the normal stress limit values may be stored in a stress database and the normal rest limit values may be stored in a rest database. Each of the normal stress and rest limit values indicates a minimum normal count value for a location within the left ventricle. Each of the normal stress limit values may be set to a mean of the count values observed in the stress MPS scan data obtained from the subjects for a location in the left ventricle minus a set threshold value (e.g., a standard deviation (“SD”) of the count values observed in the stress MPS scan data obtained from the subjects for a location in the left ventricle multiplied by a value such as 2, 2.5, 3, and the like). Each of the normal rest limit values may be may be set to a mean of the count values observed in the rest MPS scan data obtained from the subjects for a location in the left ventricle minus a set threshold value (e.g., a SD of the count values observed in the rest MPS scan data obtained from the subjects for a location in the left ventricle multiplied by a value such as 2, 2.5, 3, and the like).

Then, the count values in the patient stress and rest polar maps are compared to the normal stress and rest limit values, respectively. See Slomka P. J., Nishina H., Berman D. S., et al., Automated quantification of myocardial perfusion SPECT using simplified normal limits, J. Nucl. Cardiol., 2005; 12(1): 66-77. A count value that is lower than the applicable limit value is considered to be abnormal. The results of this comparison are then used to determine whether the patient has ischemia and in what regions of the heart ischemia is present.

Scores may be assigned to the samples (or pixels) in the patient stress and rest polar maps. For example, a score within a predetermined range (e.g., 0-4) may be assigned to each sample (or pixel) in the patient stress polar map. By way of a non-limiting example, a score within a predetermined range (e.g., 2.0 and 4.0) may indicate an abnormally low level of perfusion. For example, an abnormal score may be assigned to any samples (or pixels) having a count value below the applicable normal stress limit value for the sample (or pixel). The abnormal scores may be assigned using linear mapping based on an amount by which the count value for the sample (or pixel) is less than the normal stress limit value. A maximum abnormal score (e.g., 4.0) may be assigned to all samples (or pixels) having a count value below the applicable normal stress limit value by more than a predetermined amount (e.g., 70%). Any samples (or pixels) having a score below a predetermined minimum abnormal score (e.g., 2.0) may be reassigned a normal score (e.g., 0.0).

A total perfusion deficit (“TPD”) value may be determined for each of the scored patient stress and rest polar maps to obtain a stress TPD value and a rest TPD value, respectively. For each of the patient stress and rest polar maps, TPD may be calculated according to the following formula in which a and p are radial coordinates of the polar map, A and P are the maximum number of samples in each dimension, and score (a, p) is the sample (or pixel) score at the radial coordinates (a, p) of the polar map:

$$TPD = 100 \% \times {\sum }_{a = 0}^{a < A} {\sum }_{p = 0}^{p < P} \frac{score ( a , p )}{Max\_Score \times A \times P}$$

In other words, a TPD value is an average of the scores (assigned to the sample (or pixels)) in the polar map divided by the maximum abnormal score (e.g., 4.0). A TPD value of 100% indicates no visible radioactive tracer uptake. Research has shown a stress TPD value greater than 5% indicates the patient is likely to have CAD. See Slomka P. J., Nishina H., Berman D. S., et al., Automated quantification of myocardial perfusion SPECT using simplified normal limits, J. Nucl. Cardiol., 2005; 12(1): 66-77. Thus, the stress TPD value is a measure of hypoperfusion. Subsequently, the rest TPD value may be subtracted from the stress TPD value to estimate an amount of ischemia present in the left ventricle of the patient.

A limitation of such separate comparisons is that the unique shape of each individual heart is lost in the process, even though the shape is similar for the rest and stress MPS scans of a particular patient. Furthermore, there may be differences in orientation and position of the heart in the stress and rest MPS scan images because the images are not typically aligned. In short, when change is computed using the stress and rest TPD values, the change is in fact a derived quantity that suffers from propagation of error issues. To help overcome these problems, a general computer technique based on image co-registration of rest and stress images and voxel-by-voxel estimation of differences was proposed and described in Slomka P. J., Nishina H., Berman D. S., Kang X., Friedman J. D., Hayes S. W., Aladl U. E., Germano G., Automatic quantification of myocardial perfusion stress-rest change: a new measure of ischemia, J. Nucl. Med., 2004; 45(2): 183-91.

Because the stress and rest MPS scan images (acquired by the stress and rest MPS scans) may be obtained using different doses of radioactive tracer, at different times, and with different isotopes, a standard normalization technique (e.g., a count normalization factor) is used to normalize the stress and rest scan images. This relative nature of MPS quantification may provide another potential source of error because stress and rest count normalization factors are estimated for each pair of patient stress and rest MPS scans before samples obtained from these scans are compared to limit values stored in the stress and rest databases. Further, the normal stress and rest limit values were determined using images obtained from stress and rest MPS scans of subjects (with low likelihood of disease and visually normal scans) that were normalized using a standard normalization technique. Significant errors in standard normalization techniques have been reported. See Williams K. A., Schuster R. A., Williams K. A. Jr., Schneider C. M., Pokharna B. K., Correct spatial normalization of myocardial perfusion SPECT improves detection of multivessel coronary artery disease. J. Nucl. Cardiol. 2003; 10:353-360.

Therefore, a need exists for new methods of using stress and rest MPS scan data to detect ischemia. The present application provides this and other advantages as will be apparent from the following detailed description and accompanying figures.

### SUMMARY OF INVENTION

Aspects of the present application describe a computer implemented method for use with a patient having a heart. The method includes obtaining patient stress scan data and patient rest scan data. Then, patient stress-rest change values are determined based on the patient stress and rest scan data. After normal change limit values are obtained, whether the patient has ischemia is determined by comparing the patient stress-rest change values with the normal change limit values.

The normal change limit values may be obtained by retrieving them from a database storing the normal change limit values. Further, the normal change limit values may be obtained from a plurality of normal subjects by obtaining subject stress scan data and subject rest scan data for each of the plurality of normal subjects. Then, subject stress-rest change values based on the subject stress scan data and the subject rest scan data are obtained for each of the plurality of normal subjects. The normal change limit values are determined based on the subject stress-rest change values obtained for the plurality of normal subjects.

By way of another example, the normal change limit values may be obtained by generating a plurality of co-registered subject change polar maps for the plurality of normal subjects. The plurality of co-registered subject change polar maps comprising for each of the plurality of normal subjects may be generated by obtaining subject stress and rest scan data. A subject stress polar map comprising stress count values is generated from the subject stress scan data, the subject stress polar map. A subject rest polar map comprising rest count values is generated from the patient rest scan data. The subject stress polar map is co-registered with the subject rest polar map such that each stress count value of the subject stress polar map corresponds to a rest count value of the subject rest polar map. The stress count values are normalized with the rest count values of the co-registered subject stress and rest polar maps. Then, a subject change polar map is generated by subtracting the normalized stress count values of the subject stress polar map from the normalized rest count values of the subject rest polar map. The subject change polar map includes a plurality of polar coordinates, each associated with a subject change value. For each coordinate in the plurality of co-registered subject change polar maps, a change limit value is calculated based on the subject change values associated with the coordinate in each of the plurality of co-registered subject change polar maps.

The patient stress scan data may be obtained by performing a stress myocardial perfusion single-photon emission computerized tomography (“MPS”) scan on the patient when the patient's heart is operating under stress and the patient rest scan data may be obtained by performing a rest MPS scan on the patient when the patient's heart is operating at rest.

The method may also include generating a patient stress polar map comprising stress count values from the patient stress scan data. After normal stress limit values are obtained, a score within a predetermined range is assigned to each of the patient stress count values in the patient stress polar map based on the normal stress limit values. A patient change polar map comprising a plurality of change values is also generated from the patient stress-rest change values. Each change value in the patient rest polar map corresponds to a stress count value in the patient stress polar map. A score within the predetermined range is assigned to each of the patient stress-rest change values in the patient change polar map based on the normal change limit values, and each score assigned to the patient stress count values in the patient stress polar map compared to a first threshold value. If the score is less than the first threshold value, the score assigned to the patient stress count values in the patient stress polar map is replaced with the score assigned to the change value in the patient change polar map that corresponds to the stress count value. Then, the scores and replacement scores assigned to the stress count values in the patient stress polar map are averaged to obtain an average score. Whether the patient has ischemia may be determined by comparing the average score to a second threshold value. Further, the average score may be divided by the maximum score to obtain a percentage value, and whether the patient has ischemia may be determined by comparing the percentage value to a second threshold value.

The normal stress limit values may be obtained by retrieving them from a database storing the normal change limit values.

The patient stress-rest change values may be determined by generating a patient stress polar map comprising stress count values from the patient stress scan data, and a patient rest polar map comprising rest count values from the patient rest scan data. Then, the patient stress polar map and the patient rest polar map may be co-registered such that each stress count value of the patient stress polar map corresponds to a rest count value of the patient rest polar map. The stress count values and the rest count values of the co-registered patient stress polar map and the patient rest polar map may be normalized and the normalized stress count values of the patient stress polar map subtracted from the normalized rest count values of the patient rest polar map to obtain the patient stress-rest change values.

The patient stress-rest change values may be compared to the normal change limit values by assigning a score to each of the patient stress-rest change values based on the normal change limit values, and averaging the scores assigned to the patient stress-rest change values to obtain an average score. Then, whether the patient has ischemia may be determined by comparing the average score to a threshold value. By way of an example, the average score may be divided by the maximum score to obtain a change percentage, and whether the patient has ischemia determined by comparing the change percentage to a threshold value.

Aspects of the present application also describe a computer implemented method for use with a patient stress polar map comprising stress count values, a patient rest polar map comprising rest count values, and a normal change limit polar map comprising change limit values. The patient stress polar map is co-registered with the patient rest polar map, and the normal change limit polar map is co-registered with both the patient stress polar map and the patient rest polar map. The method includes creating a patient change polar map by subtracting the rest count values of the patient rest polar map from the stress count values of the co-registered patient stress polar map. Thus, the patient change polar map is co-registered with the normal change limit polar map. Then, the patient change polar map is compared with the normal change limit polar map to detect one or more regions in the patient change polar map in which the change value in the patient change polar map is greater that the change limit value in the co-registered normal change limit polar map. The one or more regions detected are identified as having perfusion abnormalities. The one or more regions detected as having perfusion abnormalities may be displayed to a user.

Comparing the patient change polar map with the normal change limit polar map may include assigning a score to each of the plurality of patient change values (of the patient change polar map) based at least in part on whether the patient change value is greater than the normal change limit value with which the patient change value is co-registered. The score assigned is within a predetermined range comprising a maximum score. Then, the scores assigned to the patient stress values in the patient stress polar map are averaged to obtain an average score, which is divided by the maximum score to obtain a percentage value. The method detects whether the entire patient stress polar map indicates a significant perfusion deficiency by comparing the percentage value to a threshold percentage value. By way of an example, the threshold percentage value may be between 5% and 10% and the entire patient stress polar map indicates a significant perfusion deficiency when the percentage value is greater than the threshold percentage value.

Aspects of the present application also describe a computer implemented method for use with a patient stress polar map comprising stress count values, a patient rest polar map comprising rest count values, a normal stress limit polar map comprising stress limit values, and a normal change limit polar map comprising change limit values. The patient stress polar map and the patient rest polar map are co-registered with one another. The normal change limit polar map is co-registered with both the patient stress polar map and the patient rest polar map. The normal stress limit polar map is co-registered with the patient stress polar map. The method includes creating a patient change polar map by subtracting the rest count values of the patient rest polar map from the stress count values of the co-registered patient stress polar map. The patient change polar map includes patient change values and is co-registered with the normal change limit polar map. Then the patient stress polar map is compared with the normal stress limit polar map and a score is assigned to each of the patient stress values of the patient stress polar map based on the comparison. The score assigned is a value within a predetermined range having a maximum score. For each patient stress value assigned a score below a predetermined threshold value, a patient change value is identified in the patient change polar map co-registered with the patient stress value, a normal change limit value is identified in the normal change limit polar map co-registered with the identified patient change value, a score is determined for the patient change value based on the identified normal change limit value and the identified patient change value, and the score is assigned to the patient stress value in the patient stress polar map. This score is also a value within the predetermined range. Then, whether the patient stress polar map indicates a significant perfusion deficiency is determined based on the scores assigned to the patient stress values in the patient stress polar map.

Determining whether the patient stress polar map indicates a significant perfusion deficiency may include averaging the scores assigned to the patient stress values in the patient stress polar map to obtain an average score, and dividing the average score by the maximum score to obtain a percentage value. Then, the percentage value is compared to a threshold percentage value to determine whether the patient stress polar map indicates a significant perfusion deficiency.

By way of another example, determining whether the patient stress polar map indicates a significant perfusion deficiency may include averaging the scores assigned to the patient stress values in the patient stress polar map to obtain an average score, and determining the patient stress polar map indicates a significant perfusion deficiency by comparing the average score to a threshold percentage value.

Aspects of the present application also describe one or more computer-readable media comprising instructions executable by one or more processors and when executed by the one or more processors causing the one or more processors to perform at least one of the methods described above.

Additional aspects of the present application also describe systems configured to perform at least one of the methods described above.

### BRIEF DESCRIPTION OF THE SEVERAL VIEWS OF THE DRAWING(S)

FIG. 1 is a block diagram of a system for creating and analyzing stress and rest MPS scans of a left ventricle of a heart of a patient.

FIG. 2 is a flow diagram of a method performable by the system of FIG. 1.

FIG. 3 is a block diagram illustrating data and programming modules stored in a system memory of a computing device of the system of FIG. 1.

FIG. 4A is a polar map organized by regions of the left ventricle in accordance with a 17-segment American Heart Association (“AHA”) model, each region in the polar map has been assigned a regional normal change mean value (expressed as a percentage of change) for male normal subjects only.

FIG. 4B is a polar map organized by regions of the left ventricle in accordance with a 17-segment AHA model, each region in the polar map has been assigned a regional normal change mean value (expressed as a percentage of change) for female normal subjects only.

FIG. 4C is a polar map organized by regions of the left ventricle in accordance with a 17-segment AHA model, each region in the polar map has been assigned a regional normal change mean value (expressed as a percentage of change) for both male and female normal subjects combined.

FIG. 4D is a polar map organized by regions of the left ventricle in accordance with a 17-segment AHA model, each region in the polar map has been assigned a regional normal change standard deviation (“SD”) value (expressed as a percentage of change) for male normal subjects only.

FIG. 4E is a polar map organized by regions of the left ventricle in accordance with a 17-segment AHA model, each region in the polar map has been assigned a regional normal change SD value (expressed as a percentage of change) for female normal subjects only.

FIG. 4F is a polar map organized by regions of the left ventricle in accordance with a 17-segment AHA model, each region in the polar map has been assigned a regional normal change SD value (expressed as a percentage of change) for both male and female normal subjects combined.

FIG. 5A is a graph of receiver-operating-characteristic (“ROC”) curves for change measures, which include a first receiver-operating-characteristic (“ROC”) curve for a C-SR value (labeled “C-SR”), a second ROC curve for a SDS value (labeled “SDS”), and a third ROC curve for a difference between a stress TPD value and a rest TPD value (labeled “Stress-rest TPD”).

FIG. 5B is a graph of ROC curves for combined measures, which include a first ROC curve for a stress TPD value (labeled “TPD”), a second ROC curve for a C-TPD value (labeled “C-TPD”), and a third ROC curve for a SSS value (labeled “SSS”).

FIG. 6A is bar graph of sensitivities, specificities, accuracies, and normalcy rates for change measures that include the C-SR value (labeled “C-SR”), the difference between a stress TPD value and a rest TPD value (labeled “stress TPD-rest TPD”), and the SDS value (labeled “SDS”).

FIG. 6B is bar graph of sensitivities, specificities, accuracies, and normalcy rates for combined measures that include the C-TPD value (labeled “C-TPD”), the stress TPD value (labeled “TPD”), and the SSS value (labeled “SSS”).

FIG. 7A is a collection of five two-dimensional images (three short axis images and two long axis images), each having a stress contour overlaid over the image, generated from stress MPS scan data captured from a 49 year-old female patient with single vessel disease detected by coronary angiography (80% left anterior descending (“LAD”) coronary artery stenosis).

FIG. 7B is a collection of five two-dimensional images (three short axis images and two long axis images), each having a stress contour overlaid over the image, co-registered with the images of FIG. 7A and generated from rest MPS scan data captured for the same 49 year-old female patient.

FIG. 7C is a patient stress polar map illustrating stress perfusion created using normal stress limit values and the same stress MPS scan data used to create the images of FIG. 7A.

FIG. 7D is a patient rest polar map illustrating rest perfusion created using normal rest limit values and the same rest MPS scan data used to create the images of FIG. 7B.

FIG. 7E is a patient change polar map illustrating a change in perfusion between stress and rest created using normal change limit values and the same stress and rest MPS scan data used to create the images of FIGS. 7A and 7B.

FIG. 8 is a diagram of a hardware environment and an operating environment in which the computing device of FIG. 1 may be implemented.

### DETAILED DESCRIPTION OF THE INVENTION

All publications herein are incorporated by reference to the same extent as if each individual publication or patent application was specifically and individually indicated to be incorporated by reference.

The following is a list of acronyms used in the text below:

| | |
|---|---|
| AHA | American Heart Association |
| AUC-ROC | Area Under ROC Curve |
| CAD | Coronary Artery Disease |
| CABG | Coronary Artery Bypass Graft |
| ECG | Electrocardiogram |
| LAD | Left Anterior Descending (Coronary Artery) |
| LCX | Left Circumflex (Coronary Artery) |
| LLK | Low Likelihood |
| MI | Myocardial Infarction |
| MPS | Myocardial Perfusion SPECT |
| QGS | Quantitative Gated Spect |
| RCA | Right Coronary Artery |
| ROC | Receiver-Operating-Characteristic |
| SD | Standard Deviation |
| SDS | Summed Difference Score |
| SPECT | Single-Photon Emission Computerized Tomography |
| SRS | Summed Rest Score |
| SSS | Summed Stress Score |
| TPD | Total Perfusion Deficit |

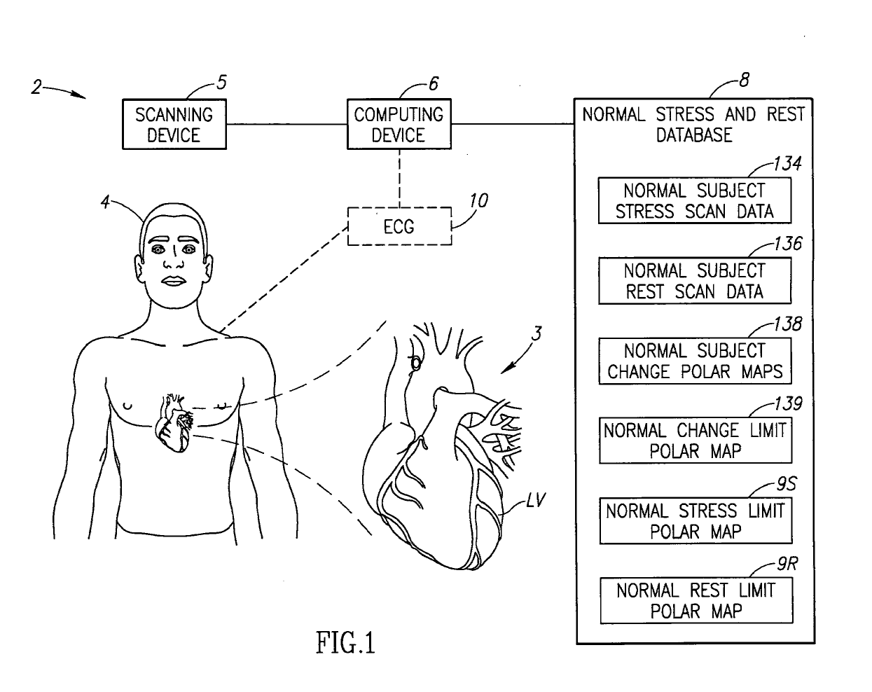

FIG. 1 is a block diagram of a system 2 for creating and analyzing stress and rest MPS scans of a left ventricle “LV” of a heart 3 of a patient 4. The system 2 includes a scanning device 5 configured to perform stress and rest MPS scans used to obtain stress and rest scan data, respectively. The stress and rest scan data is analyzed by a computing device 6 connected to the scanning device 5. A database 8 accessible by the computing device 6 stores normal stress and rest limit values. By way of a non-limiting example, the normal stress and rest limit values may be calculated by the computing device 6 and/or the database 8. The normal stress and rest limit values may be stored as or used to construct normal stress and rest limit polar maps 9S and 9R, respectively. While illustrated as separate from and connected to the computing device 6, in alternate implementations, the database 8 may be stored by the computing device 6. Depending upon the implementation details, the database 8 may be implemented by one or more computing devices substantially similar to the computing device 6.

The normal stress limit values may be obtained from stress MPS scans performed on subjects (with low likelihood of disease and visually normal scans) and used to create the normal stress limit polar map 9S (stored in the database 8) in which each sample is associated with a stress limit value indicating a minimum amount of normal stress perfusion and a location value identifying a location within the left ventricle from which the stress limit value is believed to have been obtained. Similarly, normal rest limit values may be obtained from rest MPS scans performed on subjects (with low likelihood of disease and visually normal scans) and used to create the normal rest limit polar map 9R (stored in the database 8) in which each sample is associated with a rest limit value indicating a minimum amount of normal rest perfusion and a location value identifying a location within the left ventricle from which the rest limit value is believed to have been obtained.

An optional electrocardiogram (“ECG”) 10 may be connected to the patient 4 in a conventional manner and used to detect a cardiac cycle of the heart 3. The optional ECG 10 may also be connected to the computing device 6 and/or the scanning device 5 and used to time when scans are performed by the scanning device 5. By way of a non-limiting example, the ECG 10 may be configured to transmit a signal to the computing device 6 representing the cardiac cycle. The computing device 6 may analyze the signal to detect particular points in the cardiac cycle and direct the scanning device 5 to perform a scan based on the detection of these particular points in the cardiac cycle. This process is commonly referred as gated SPECT. Thus, instead of obtaining a single stress MPS scan and a single rest MPS scan, the scanning device 5 may obtain multiple stress and rest MPS scans.

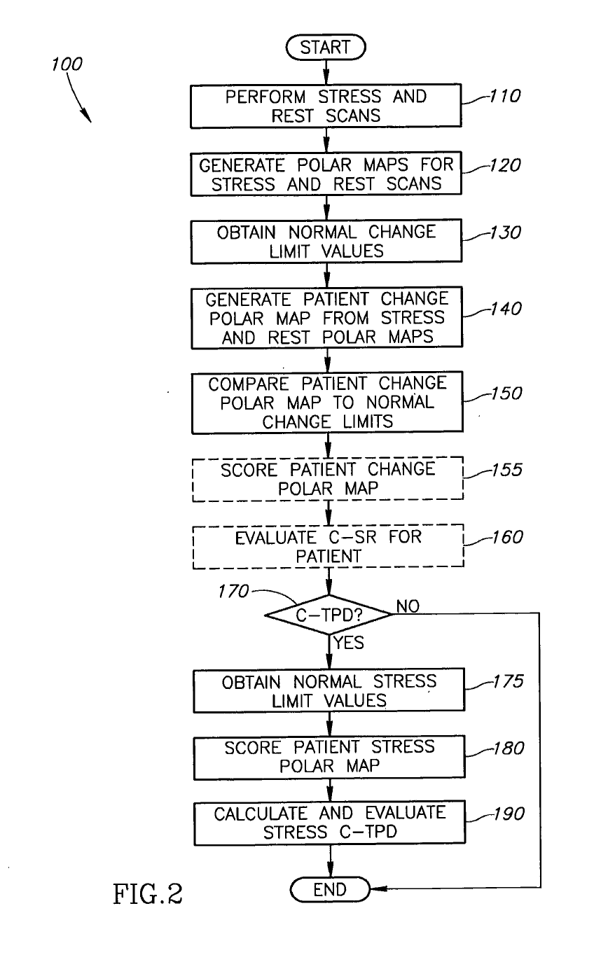

A method 100 that may be performed by the system 2 to detect and evaluate hypoperfusion in the left ventricle “LV” of the patient's heart 3 will now be described with respect to FIGS. 1 and 2. In first block 110, the scanning device 5 performs at least one conventional stress MPS scan and at least one conventional rest MPS scan of the left ventricle “LV” of the patient's heart 3. Each stress MPS scan is performed when the heart 3 is operating under stress (e.g., caused by exercise, induced chemically, and the like) and each rest MPS scan is performed when the heart 3 is operating at rest. Patient stress scan data is captured by the scanning device 5 when it performs the at least one conventional stress MPS scan and patient rest scan data is captured by the scanning device 5 when it performs the at least one conventional rest MPS scan.

Methods of performing conventional stress and rest MPS scans are known in the art and will not be described in detail. As mentioned above, multiple stress and rest MPS scans may be performed and gated using the ECG 10 to guide or time the acquisition of scan data.

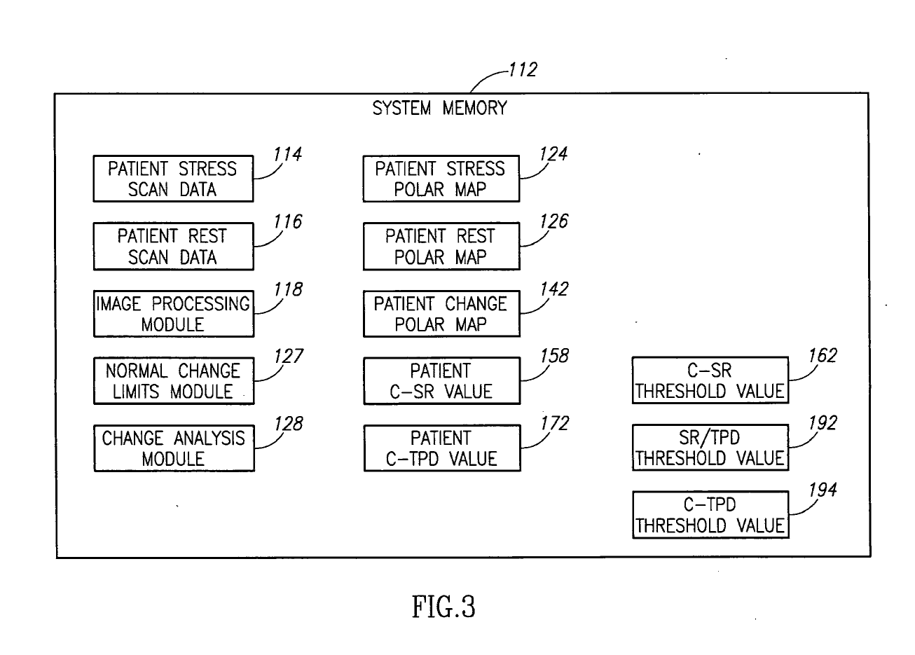

The patient stress scan data and patient rest scan data captured in block 110 is transferred to the computing device 6 for analysis. FIG. 3 is an illustration of at least a portion of the data and programming modules stored in a system memory 112 of the computing device 6. In FIG. 3, patient stress scan data 114 and patient rest scan data 116 are both illustrated as being stored in the system memory 112.

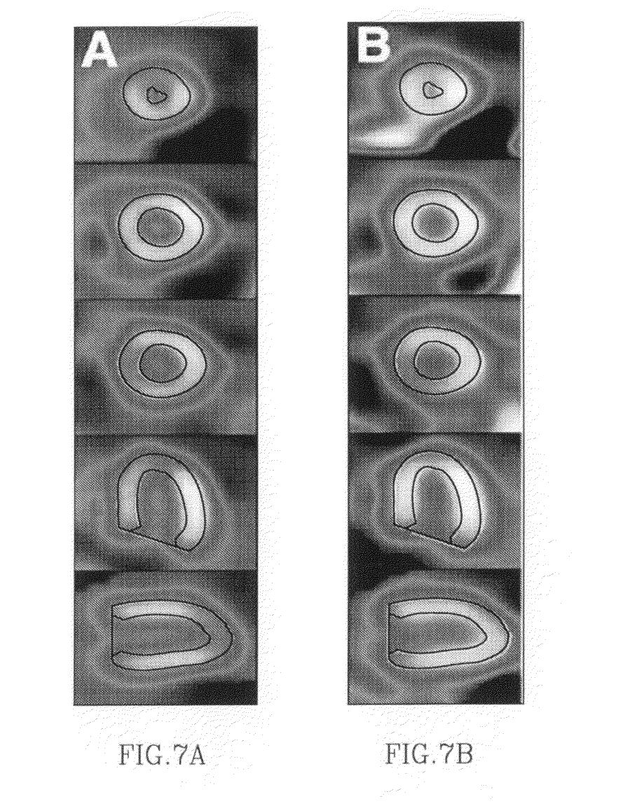

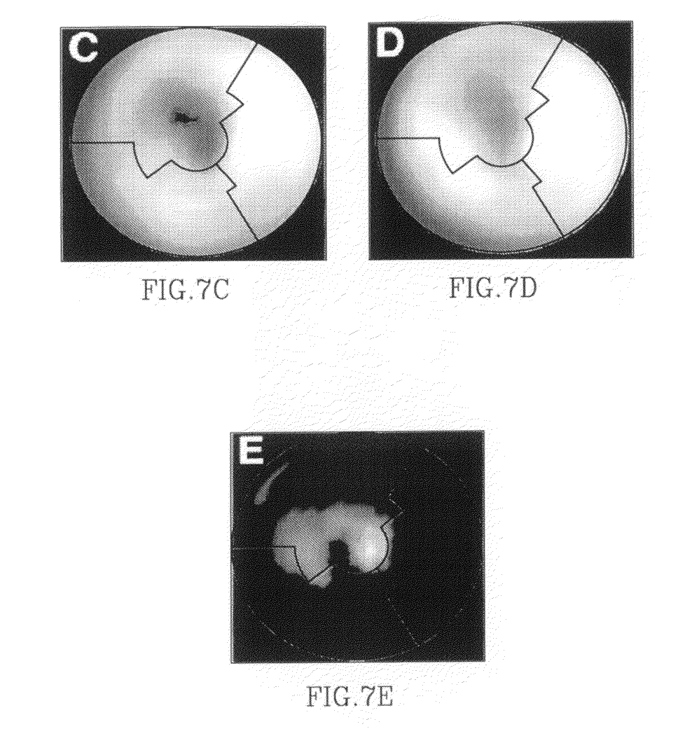

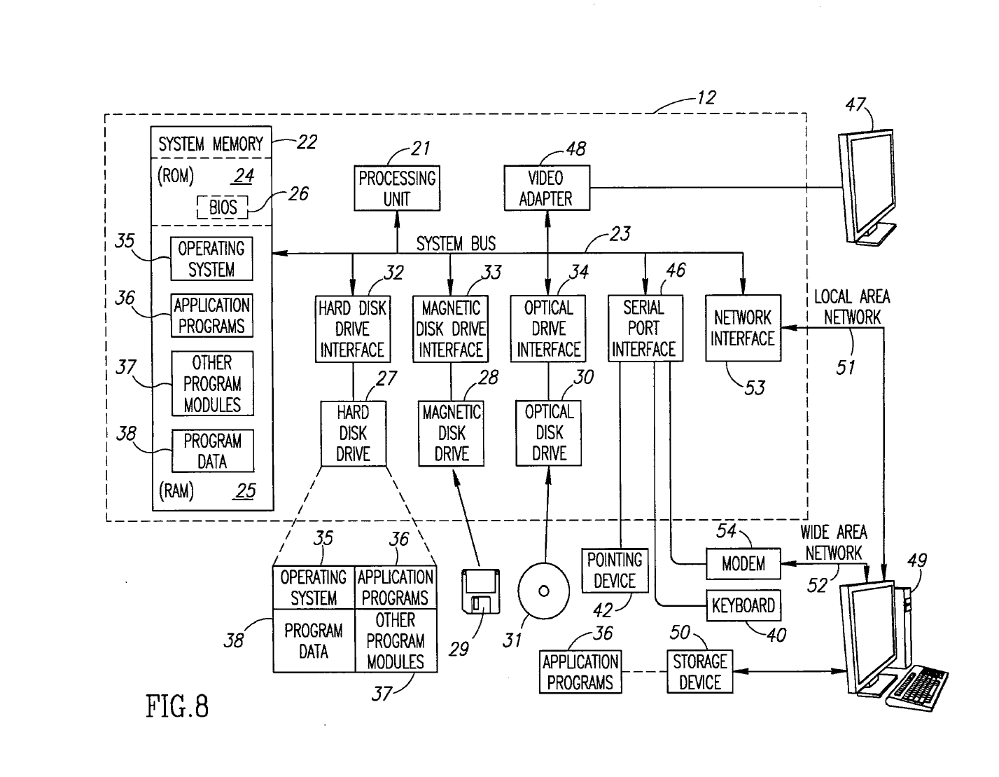

As explained in the Background Section, for each MPS scan, the computing device 6 may construct a three-dimensional representation of the scan data. The system memory 112 stores an image processing module 118 having computer-executable instructions that when executed by one or more processors (e.g., a processing unit 21 illustrated in FIG. 8) are operable to construct a three-dimensional representation of at least a portion of the left ventricle “LV” from the scan data. Further, the image processing module 118 may have instructions that when executed by one or more processors (e.g., the processing unit 21 illustrated in FIG. 8) are operable to generate two-dimensional representations (e.g., images or slices) of the scan data from the three-dimensional representation. For example, the image processing module 118 may include instructions for sampling the three-dimensional representation to produce two-dimensional images. FIG. 7A provides examples of two-dimensional images generated by the instructions of the image processing module 118 from the patient stress scan data 114, and FIG. 7B provides examples of two-dimensional images generated by the instructions of the image processing module 118 from the patient rest scan data 116.

The image processing module 118 may have instructions that when executed by one or more processors (e.g., the processing unit 21 illustrated in FIG. 8) are operable to construct patient stress and rest polar maps 124 and 126 based on the patient stress and rest scan data 114 and 116, respectively. Returning to FIGS. 1-3, in block 120, the computing device 6 (executing the image processing module 118) constructs the patient stress and rest polar maps 124 and 126 based on the patient stress and rest scan data 114 and 116, respectively. By way of a non-limiting example, a summed patient stress polar map may be created using all of scan data captured by the stress MPS scan(s) performed on the patient 4. Alternatively, a separate patient stress polar map may be created for each stress MPS scan performed on the patient 4. Thus, a separate stress polar map may be created for each point in the cardiac cycle for which a stress MPS scan was performed on the patient 4. Similarly, a summed patient rest polar map may be created using all of images captured by the rest MPS scan(s) performed on the patient 4. Alternatively, a separate patient rest polar map may be created for each rest MPS scan performed on the patient 4. Thus, a separate rest polar map may be created for each point in the cardiac cycle for which a rest MPS scan was performed on the patient 4.

For ease of illustration, the method 100 will be described with respect to the single patient stress polar map 124 and the single patient rest polar map 126. However, through application of ordinary skill to the present teachings, embodiments may be practiced with multiple patient stress polar maps and multiple patient rest polar maps. Therefore, such embodiments are within the scope of the present teachings.

Each of the patient stress and rest polar maps 124 and 126 is a two-dimensional representation perfusion in the left ventricle. By way of non-limiting examples, FIG. 7C provides an example of a patient stress polar map and FIG. 7D provides an example of a patient rest polar map. The patient stress and rest polar maps 124 and 126 may be displayed on a conventional computer monitor (e.g., a monitor 47 illustrated in FIG. 8) and when so displayed, may each be described as including pixels. As explained in the Background Section, each sample used to construct the patient stress and rest polar maps 124 and 126 is associated with (1) a count value indicating an amount of perfusion (e.g., an amount of radioactive tracer detected) and (2) a location value identifying a location within the left ventricle “LV” from which the count value is believed to have been obtained. The count value indicates density of the radioactive tracer at the location value. Each pixel may correspond to one or more of the samples used to construct the patient stress and rest polar maps 124 and 126. Alternatively, a sample may be represented by more than one pixel. If a pixel corresponds to a single sample, the pixel may be assigned the count value of the sample. If a pixel corresponds to more than one sample, the pixel may be assigned an aggregated count value determined based on a combination (e.g., an average) of the count values of the samples represented by the pixel. If a sample is represented by more than one pixel, each pixel may be assigned the count value of the sample.

Thus, the analyses performed by the computing device 6 may be performed on pixels, samples, and the like. For ease of illustration, such analyses will be described as being performed on samples. However, through application of ordinary skill in the art to the present teachings, embodiments in which such analyses are instead performed on pixels or other values derived from the samples may be constructed. Therefore, such embodiments are within the scope of the present teachings.

In block 130, the computing device 6 obtains normal change limit values. The system memory 112 of the computing device 6 includes a normal change limits analysis module 127 having computer-executable instructions that when executed by one or more processors (e.g., the processing unit 21 illustrated in FIG. 8) are operable to obtain the normal change limit values. The database 8 may include normal subject stress scan data 134 obtained from stress MPS scans performed on subjects (with low likelihood of disease and visually normal scans) and normal subject rest scan data 136 obtained from rest MPS scans performed on subjects (with low likelihood of disease and visually normal scans). The normal change limits analysis module 127, when executed by one or more processors, generates change polar maps 138 from the normal subject stress and rest scan data 134 and 136. For example, a stress polar map may be created for each subject using the normal subject stress scan data 134 and a rest polar map may be created for each subject using the normal subject rest scan data 136. Then, for each subject, the stress count values in the stress polar map may be normalized with the rest count values in the rest polar map. For each subject, a normal change map may be created by subtracting the normalized rest count values in the subject's rest polar map from the normalized stress count values in the subject's stress polar map.

For each subject, to create the normal change map for the subject, it may be necessary to co-register and normalize the stress and rest polar maps obtained for the subject. Thus, each sample (a, p) in the subject's stress polar map has the same address as a corresponding sample (representing the same portion of the left ventricle) in the subject's rest polar map. Then, the rest count values in the subject's rest polar map may be subtracted from the stress count values of the corresponding samples in the subject's stress polar map to create the change polar map for the subject. Each sample in the subject's change polar map is associated with a change value (i.e., the stress count value—the rest count value) and a location in the left ventricle of the subject from which the stress and rest count values are believed to have been obtained.

After change polar maps 138 (see FIG. 1) have been generated for the subjects, the normal change limit values may be determined using these change polar maps. For example, the change values in the change polar maps 138 may be averaged to obtain the normal change limit values. The normal change limit values may be used to construct a normal change limit polar map 139 having the same coordinates as the subjects' change polar maps 138. In the normal change limit polar map 139, each coordinate (a, p) is associated with a normal change limit value and a location in the left ventricle.

By way of a non-limiting example, the normal change limit values may be determined by combining corresponding change values (i.e., change values having the same coordinates (a, p)) in the subjects' change polar maps 138. A normal change mean value and a normal change standard deviation (“SD”) value may be determined for each corresponding change value (a, p) in the subjects' change polar maps 138. Because a large change between stress and rest perfusion indicates ischemia, a change value observed between the patient's stress and rest MPS scans that is significantly greater than a corresponding normal change mean value (at the same location in the left ventricle) may be used to identify ischemia. Thus, the normal change limit values for each coordinate (a, p) in the normal change limit polar map 139 may be set to a sum of the normal change mean value at the same coordinate (a, p) in the subjects' change polar maps 138 and twice the normal change SD value at the same coordinate (a, p) in the subjects' change polar maps 138.

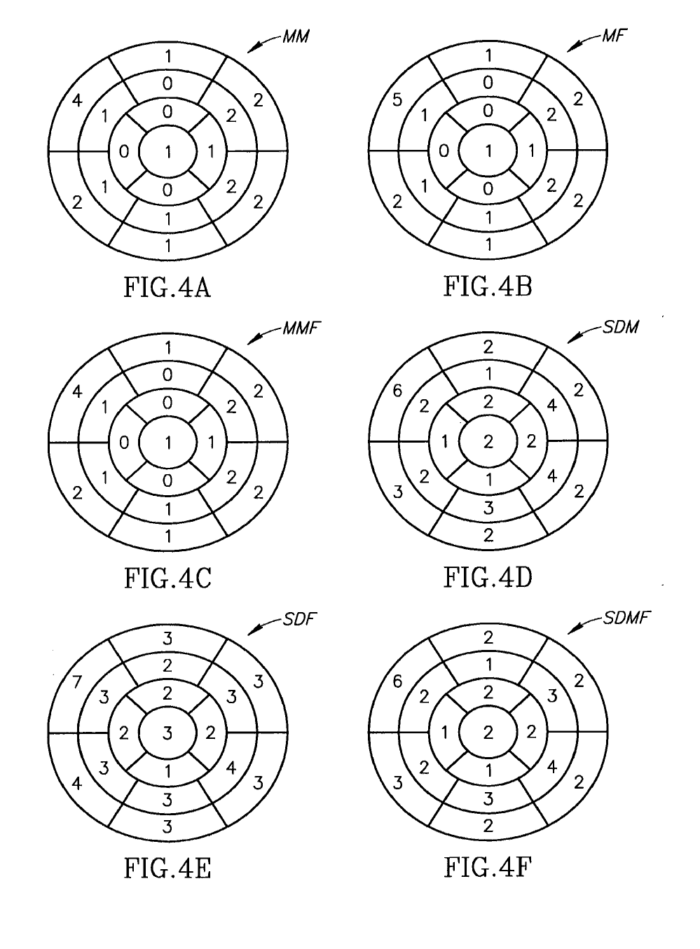

Alternatively, a regional normal change mean value and a regional normal change SD value may be determined for each of a plurality of regions in the subjects' change polar maps. FIGS. 4A-4C provide three exemplary polar maps “MM,” “MF,” and “MMF,” each organized by regions of the left ventricle in accordance with a 17-segment American Heart Association (“AHA”) model. In the polar map “MM,” each region has been assigned the regional normal change mean value (expressed as a percentage of change) determined for the region for male normal subjects only. In the polar map “MF,” each region has been assigned the regional normal change mean value (expressed as a percentage of change) determined for the region for female normal subjects only. In the polar map “MMF,” each region has been assigned the regional normal change mean value (expressed as a percentage of change) determined for the region for male and female normal subjects combined. FIG. 4D-4F provides three exemplary polar maps “SDM,” “SDF,” and “SDMF,” each also organized by regions of the left ventricle in accordance with the 17-segment AHA model. In the polar map “SDM,” each region has been assigned the regional normal change SD value (expressed as a percentage of change) determined for the region for male normal subjects only. In the polar map “SDF,” each region has been assigned the regional normal change SD value (expressed as a percentage of change) determined for the region for female normal subjects only. In the polar map “SDMF,” each region has been assigned the regional normal change SD value (expressed as a percentage of change) determined for the region for male and female normal subjects combined.

A regional normal change limit value may be determined for each region and used to construct a regional normal change limit polar map (not shown). A regional change value observed between the patient's stress and rest MPS scans that is significantly greater than a corresponding regional normal change mean value (for the same region in the left ventricle) may be used to identify ischemia. Thus, regional normal change limit values may be set to a sum of the regional normal change mean value and twice the regional normal change SD value determined for the region.

Because the normal change limit polar map may include a normal change limit value for each pixel, sample, region, and the like, each of which may be addressed by a unique polar coordinate (a, p), the normal change limit polar map will be described as having coordinates that are each associated with a normal change limit value for a portion of the left ventricle.

The system memory 112 of the computing device 6 includes a change analysis module 128 having computer-executable instructions that when executed by one or more processors (e.g., the processing unit 21 illustrated in FIG. 8) are operable to generate a patient change polar map 142. In block 140, the computing device 6 (executing the change analysis module 128) generates the patient change polar map 142. To generate the patient change polar map 142, it may be necessary to co-register and normalize the patient stress and rest polar maps 124 and 126. Thus, each sample (a, p) in the patient stress polar map 124 has the same address as a corresponding sample (representing the same portion of the left ventricle “LV”) in the patient rest polar map 126. Then, the count values in the patient rest polar map 126 may be subtracted from the count values of the corresponding samples in the patient stress polar map 124 to create the patient change polar map 142. Each sample in the patient change polar map 142 is associated with a change value (i.e., the stress count value—the rest count value) and a location in the left ventricle “LV” of the patient 4 from which the stress and rest count values are believed to have been obtained.

Optionally, the change values associated with the samples in the patient change polar map 142 may be expressed as a percentage of change between stress and rest. Further, the change values may be combined to express change within a region of the left ventricle “LV.” For example, the change values within a region may be averaged (e.g., averaged) or otherwise combined to create an aggregated change value. The regions may be defined in accordance with a predetermined standard such as the 17-segment AHA model.

Because the patient change polar map 142 may include a change value for each pixel, sample, region, and the like, each of which is addressed by a unique polar coordinate (a, p), the patient change polar map 142 will be described as having coordinates that are each associated with a change value for a portion of the left ventricle “LV” of the patient's heart 3.

The change analysis module 128 has computer-executable instructions that when executed by one or more processors (e.g., the processing unit 21 illustrated in FIG. 8) are operable to compare the change values in the patient change polar map 142 to the normal change limit values in the normal change limit polar map 139. In block 150, the computing device 6 (executing the change analysis module 128) compares the change values in the patient change polar map 142 to the normal change limit values in the normal change limit polar map 139. To compare the patient change polar map 142 to the normal change limit polar map 139, it may be necessary to co-register and normalize the patient change polar map 142 and normal change limit polar map 139. Thus, each coordinate (a, p) in the patient change polar map 142 corresponds to a coordinate (a, p) in the normal change limit polar map 139 having a normal change limit value for the same location in the left ventricle associated with the coordinate (a, p) in the patient change polar map 142. Any areas of the patient change polar map 142 having change values that exceed corresponding normal change limit values in the normal change limit polar map 139 may be characterized as having perfusion abnormalities.

The change analysis module 128 has computer-executable instructions that when executed by one or more processors (e.g., the processing unit 21 illustrated in FIG. 8) are operable to assign a score within a predetermined range (e.g., 0-4) to each coordinate in the patient change polar map 142 based on the normal change limit values in the normal change limit polar map 139. In optional block 155, the computing device 6 (executing the change analysis module 128) assigns a score within a predetermined range (e.g., 0-4) to each coordinate in the patient change polar map 142 based on the normal change limit values in the normal change limit polar map 139. By way of a non-limiting example, a score within a predetermined range (e.g., 2.0 and 4.0) may be considered abnormal indicating poor profusion into the muscles of the heart. Such abnormal scores may be assigned to any coordinate in the patient change polar map 142 having a change value that is greater than the normal change limit in the normal change limit polar map 139 for the coordinate. The abnormal scores may be assigned (e.g., using linear mapping) based on an amount by which the change value is greater than the normal change limit. A maximum abnormal score (e.g., 4.0) may be assigned to all coordinates more than a predetermined amount (e.g., 70%) above the applicable normal change limit. Any coordinates assigned a score below a minimum abnormal score (e.g., 2.0) may be reassigned a normal score (e.g., 0.0) indicating the change in profusion is normal at the location of the heart 3 associated with the coordinate.

The change analysis module 128 may include computer-executable instructions that when executed by one or more processors (e.g., the processing unit 21 illustrated in FIG. 8) are operable to calculate a global patient stress-rest perfusion change “C-SR” value 158 based on the patient change polar map 142. In optional block 160, the computing device 6 (executing the change analysis module 128) calculates the “C-SR” value 158. By way of a non-limiting example, in optional block 160, the computing device 6 may calculate the C-SR value 158 by integrating (or summing) the individual scores assigned to the coordinates in the patient change polar map 142. A formula substantially similar to the one used to calculate TPD (described in the Background Section) may be used to calculate the C-SR value 158 for the patient change polar map 142. The computing device 6 (executing the change analysis module 128) may compare the C-SR value 158 to a C-SR threshold value 162 and based on this comparison determine whether the patient has myocardial ischemia, CAD, and the like. For example, if the C-SR value 158 exceeds the C-SR threshold value 162 (e.g., 5%, 10%, etc.), the computing device 6 may indicate the patient has an abnormal scan with ischemia indicating presence of CAD.

The change analysis module 128 has computer-executable instructions that when executed by one or more processors (e.g., the processing unit 21 illustrated in FIG. 8) are operable to calculate a “C-TPD” value 172. In decision block 170, a decision is made as to whether to calculate the “C-TPD” value 172. The decision in decision block 160 may be made by an operator of the computing device 6. Therefore, in block 170, the computing device 6 may receive a command or instruction from the operator via a user interface (described below) to calculate the “C-TPD” value 172. In such embodiments, the decision in decision block 160 is “YES” when the computing device 6 receives a command or instruction to calculate the “C-TPD” value 172. On the other hand, the decision in decision block 160 is “NO” when the computing device 6 does not receive a command or instruction to calculate the “C-TPD” value 172. When the decision in decision block 170 is “YES,” block 175 is performed. When the decision in decision block 170 is “NO,” the method 100 terminates.

To calculate the “C-TPD” value 172, the patient change polar map 142 must be scored. Therefore, block 155 must be performed if it was not performed earlier.

In block 175, the computing device 6 obtains the normal stress limit values. By way of a non-limiting example, the normal stress limit values may be obtained from the database 8. As explained above, the normal stress limit values may be arranged in the normal stress limit polar map 9S.

In block 180, the computing device 6 (executing the change analysis module 128) assigns a score to the samples of the patient stress polar map 124. To assign scores to the samples of the patient stress polar map 124, it may be necessary to co-register and normalize the normal stress limit polar map 9S and the patient stress polar map 124. Thus, each sample (a, p) in the patient stress polar map 124 has the same address as a corresponding coordinate (representing the same portion of the left ventricle) in the normal stress limit polar map 9S. For example, as with a conventional stress TPD determination, a score within a predetermined range (e.g., 0-4) may be assigned to each sample in the patient stress polar map 124. By way of a non-limiting example, a score within a predetermined range (e.g., 2.0 and 4.0) may indicate an abnormally low level of perfusion. For example, an abnormal score may be assigned to any sample in the patient stress polar map 124 having a count value that is less than the applicable normal stress limit value (in the normal stress limit polar map) corresponding to the sample. The abnormal scores may be assigned (e.g., using linear mapping) based on an amount by which the count value is less than the normal change limit. A maximum abnormal score (e.g., 4.0) may be assigned to all samples having a count value less than the applicable normal stress limit value by more than a predetermined amount (e.g., 70%). Any samples having a score below the minimum abnormal score (e.g., 2.0) may be reassigned a normal score (e.g., 0.0).

In block 190, the computing device 6 (executing the change analysis module 128) determines the C-TPD value 172 for the patient stress polar map 124. By way of a non-limiting example, the C-TPD value 172 may be determined using a sample-by-sample analysis. For each sample in the patient stress polar map 124, the score (or measure of stress perfusion abnormality) assigned to the sample may be compared to a SR/TPD threshold value 192 (e.g., 2.0). If the score is less than the SR/TPD threshold value 192, the score previously assigned to the sample is replaced with the score assigned to the corresponding sample in the patient change polar map 142 in block 155. Then, the C-TPD value 172 is calculated by totaling the scores assigned to the samples in the patient stress polar map 124. The rationale for this approach is that the change value may be better at detecting subtle hypoperfusion defects than the results of the comparison of the stress count value and the normal stress limit value.

In block 190, the computing device 6 may compare the C-TPD value 172 to a C-TPD threshold value 194. The computing device 6 (executing the change analysis module 128) may use the results of this comparison to determine whether the patient has myocardial ischemia. For example, if the C-TPD value 172 exceeds the C-TPD threshold value 194 (e.g., 5%, 10%, etc.), the patient 4 may be diagnosed with a high likelihood of CAD.

Blocks 130-190 of the method 100 may be repeated for each interval of or point in the cardiac cycle for which MPS scans were performed and polar maps generated.

A study was conducted using an embodiment of the method 100.

### Study

The study was a retrospective one and Institutional Review Board approval was obtained. The total study population consisted of 997 patients who underwent exercise or adenosine stress technetium-99m (“^{99m}Tc”) sestamibi MPS. Characteristics of these patients are provided in Table 1 below. To obtain normalcy rates, 346 consecutive patients with a low likelihood (“LLK”) of CAD were analyzed.

TABLE 1

| Parameter | Angiography Value | LLK Value |
|---|---|---|
| Age (y) | 64 ± 12 | 52 ± 11 |
| Sex (female) | 282 (43%) | 218 (63%) |
| BMI | 31 ± 6 | 29 ± 6 |
| Hypertension | 414 (63%) | 134 (39%) |
| Hypercholesterolemia | 273 (42%) | 184 (53%) |

For angiographic validation, 651 consecutive patients (369 males, 282 females) who had a coronary angiography within three months of the MPS scans were used. Exclusion criteria were as follows: (a) prior myocardial infarction (“MI”) or coronary revascularization; (b) non-ischemic cardiomyopathy or vascular heart disease; and (c) change in symptoms between MPS and coronary angiography. A distribution of diseased vessels in the angiographic population (n=651) is provided in Table 2.

TABLE 2

| Category | ≧70% stenosis | ≧50% |
|---|---|---|
| 0 vessel (no disease) | 222 | 184 |
| 1 vessel | 232 | 192 |
| 2 vessels | 127 | 151 |
| 3 vessels | 70 | 124 |

Normal stress and rest limit values were evaluated from a separate group of 80 normal subjects (40 women and 40 men) that were selected consecutively with a LLK of CAD (less than 5%) based on age, gender, pretest symptoms, and ECG response to adequate treadmill stress testing.

The number of diseased left anterior descending (“LAD”) coronary artery vessels, left circumflex (“LCX”) coronary artery vessels, and right coronary artery (“RCA”) vessels (defined as having greater than or equal to 70% lesion) were 280, 169, and 247, respectively. The number of diseased LAD and LCX, LAD and RCA, and LCX and RCA vessels (defined as having greater than or equal to 70% lesion) were 101, 138, and 98, respectively. Seventy cases of triple-vessel disease were present in our dataset when using greater than or equal to 70% stenosis as a criterion.

Studies were performed using ^{99m}Tc rest and ^{99m}Tc stress protocols. A same-day rest/stress protocol was used for women who weighed less than 200 lb or whose BMI was less than 35 kg/m² and for men who weighed less than 250 lb or whose BMI was less than 40 kg/m². A two-day rest/stress or stress/rest protocol was used for those individuals whose weight or BMI levels were above these levels. The weight-BMI-related ^{99m}Tc sestamibi dose ranged from 8.5 mCi to 11.6 mCi for rest MPS scans to 29.5 mCi to 42 mCi for stress MPS scans. For two-day protocols, the “stress” dose was used for both the rest and stress portions of the study.

The details of image acquisition and tomographic reconstruction for this study were substantially identical to those described in Slomka P. J., Fish M. B., Lorenzo S., et al. Simplified normal limits and automated quantitative assessment for attenuation-corrected myocardial perfusion SPECT, J. Nucl. Cardiol, September 2006; 13(5):642-651. In brief, all subjects were first imaged (a) 60 minutes after the administration of ^{99m}Tc sestamibi at rest, or (b) after 60 minutes of and during adenosine infusion with the subject at rest. The subjects were then imaged again 15 to 45 minutes after either (a) radiopharmaceutical injection during treadmill testing, or (b) adenosine infusion with low-level exercise.

MPS scans of each subject and patient were acquired using Vertex, dual-detector scintillation cameras with low energy high-resolution collimators (Vertex, Philips Medical Systems). For this analysis, attenuation-corrected data was not used. All acquisitions were performed with a noncircular 180° orbit, from 45° right anterior oblique to the left posterior oblique, with a 64×64 matrix (pixel size=0.64 cm). At each of the 64 projection angles, the image data were recorded in eight equal ECG gated time bins. The time per projection used in this study was 45 to 50 seconds for rest MPS scans, and 30 to 40 seconds for stress MPS scans.

Rest and stress doses of radioactive tracer were administered using a weight-related scale and ranged from 8-12 mCi for rest and 30-42 mCi for stress.

Tomographic reconstruction was performed by use of the AutoSPECT and Vantage Pro programs (Philips Medical Systems).

Coronary angiography was performed with the standard Judkins approach, and all coronary angiograms were interpreted visually by a physician with more than 30 years of experience with myocardial perfusion studies. The arbitrary cutoff point used for the definition of CAD is greater than 70% narrowing of maximal lumen diameter.

A LLK of CAD (less than 5%) was defined based on age, sex, pretest symptoms, and electrocardiogram response to treadmill stress testing. See Diamond G. A. and Forrester J. S., Analysis of probability as an aid in the clinical diagnosis of coronary-artery disease, N. Engl. J. Med. 1979; 300: 1350-1358. Accordingly, subjects who underwent treadmill stress testing and who had an adequate level of treadmill stress (greater than 85% of predicted maximum heart rate) were chosen. These subjects had no history of CAD (a previous MI or coronary revascularization) or other confounding cardiac conditions, including congestive heart failure, cardiomyopathy, significant vascular or congenital heart disease, left-bundle branch block, or paced rhythm. These subjects did not undergo coronary angiography. Furthermore, these subjects had MPS studies of good to excellent quality, normal ventricular volumes (as described in Sharir T., Kang X., Shaw L. J., Gransar H., Cohen I., Germano G., Hayes S. W., Friedman J. D., Berman D. S, and Bax J. J., Prognostic value of poststress left ventricular volume and ejection fraction by gated myocardial perfusion SPECT in women and men: Gender-related differences in normal limits and outcomes, J. Nucl. Cardiol., 2008; 13 (4): 495-506), normal wall motion, and normal global systolic function, and no evidence of transient ischemic dilation, as judged by the director of the MPS laboratory where the data were acquired.

Left ventricle extraction and fitting to an ellipsoidal model using the Quantitative Gated SPECT (“QGS”) algorithm was performed to derive polar map representations as previously described in Germano G., Kiat H., Kavanagh P. B., et al., Automatic quantification of ejection fraction from gated myocardial perfusion SPECT, J. Nucl. Med. 1995; 36:2138-2147. Count normalization was implemented using an iterative scheme, as previously performed for stress-rest image normalization in Slomka P. J., Nishina H., Berman D. S., et al., Automatic quantification of myocardial perfusion stress-rest change: a new measure of ischemia, J. Nucl. Med. 2004; 45:183-191. All results were derived using batch mode processing without human intervention of the algorithms described with respect to this study. The algorithms were applied to the already reconstructed short axis data out of which 124 cases had contours corrected.

As previously described Slomka P. J., Nishina H., Berman D. S., et al., Automatic quantification of myocardial perfusion stress-rest change: a new measure of ischemia, J. Nucl. Med., 2004; 45:183-191, stress and rest TPD values each combine defect severity and extent in one parameter. For the purposes of this study, a standard measure of change was defined as a difference between the stress TDP value and the rest TPD value (“stress TPD-rest TPD”) as currently utilized and described in Shaw L. J., Berman D. S., Maron D. J. et al., Optimal medical therapy with or without percutaneous coronary intervention to reduce ischemic burden: results from the Clinical Outcomes Utilizing Revascularization and Aggressive Drug Evaluation (COURAGE) trial nuclear substudy, Circulation, 2008; 117(10): 1283-91.

In addition to pixel-based quantitative measurements, a computed 17-segment summed stress score (“SSS”) value and a summed difference score (“SDS”) value were used as bases for comparison. Methods for calculating the SSS and SDS values from polar map samples are provided in Slomka P. J., Nishina H., Berman D. S., et al., Automated quantification of myocardial perfusion SPECT using simplified normal limits, J. Nucl. Cardiol., 2005; 12(1): 66-77

For each of the LLK of CAD subjects (40 males and 40 females) and test patients, pairs of stress and rest images were co-registered and normalized to each other as previously described in Slomka P. J., Nishina H., Berman D. S., et al. Automatic quantification of myocardial perfusion stress-rest change: a new measure of ischemia, J. Nucl. Med., 2004; 45:183-191.

The normal database (e.g., the database 8) contained a case for each of the LLK of CAD subjects. Change polar maps were then generated for each case and stored in the normal database. The normal database thus contained change values for each radial coordinate (a, p) of the polar map corresponding to each LLK of CAD subject. Upper normal change limit values (two standard deviations above the mean) were then established for each radial coordinate (a, p).

Change polar maps were also generated for each test subject. Subsequently, the global C-SR value was calculated by integrating individual changes for each polar map pixel after (scoring or) scaling each change pixel to standard 0-4 scale as previously described for TPD calculations in Slomka P. J., Nishina H., Berman D. S., et al., Automated quantification of myocardial perfusion SPECT using simplified normal limits, J. Nucl. Cardiol., 2005; 12(1): 66-77.

Additionally, the C-TPD value was calculated for each test patient using an empiric rule applied to each polar map pixel to combine the global C-SR value with the stress TPD value. For each polar map pixel (in the patient stress polar map), when stress perfusion abnormality (i.e., the score assigned to the pixel based on the stress count value as compared to normal stress limit value) fell below a certain threshold value (e.g., 2.0), the stress hypoperfusion value (the score assigned to the pixel for the TPD calculation) was replaced with the corresponding score of the change value (which was also within the scale from 0 to 4) for the pixel. The rationale for this approach is that subtle hypoperfusion defects may be better detected by change analysis than by comparison to normal stress limit values. Using a step size of 0.5, candidate threshold values within the range (e.g., 0-4) of the scores were tested, with a threshold value of 2.0 resulting in the highest area under the receiver-operating-characteristic (“AUC-ROC”) for the detection of CAD from MPS scan data in the study.

In the statistical analysis, all continuous variables are expressed as mean±SD. Paired t-tests were used to compare differences in paired continuous data and McNemar tests were used to compare differences in paired discrete data. A P-value (“P”) of less than 0.05 was considered significant. The receiver-operating-characteristic (“ROC”) curve analysis was performed to evaluate the ability of the quantification to predict greater than 70% stenosis of coronary arteries. In all analysis, the absence of CAD was defined as LLK of disease or less than 70% stenosis in angiography cases. Both groups were combined and considered as normal as previously suggested avoiding bias and providing a balanced set of data with approximately 50% of cases abnormal for subsequent analysis. See Slomka P. J., Fish M. B., Lorenzo S., et al. Simplified normal limits and automated quantitative assessment for attenuation-corrected myocardial perfusion SPECT, J. Nucl. Cardiol., September 2006; 13(5):642-651.

### Results of Study

In FIGS. 4A-4G, the average and SD of the stress-rest changes obtained from the normal database (40 males and 40 females) are displayed using the 17-segment AHA model. The polar map “MM” in FIG. 4A depicts the mean of the stress-rest changes for the normal male subjects only. The polar map “MF” in FIG. 4B depicts the mean of the stress-rest changes for the normal female subjects only. The polar map “MMF” in FIG. 4C depicts the mean of the stress-rest changes for all normal subjects combined (i.e., gender-combined). The polar map “SDM” in FIG. 4D depicts the SD of the stress-rest changes for the normal male subjects only. The polar map “SDF” in FIG. 4E depicts the SD of the stress-rest changes for the normal female subjects only. The polar map “SDMF” in FIG. 4F depicts the SD of the stress-rest changes for all normal subjects combined (i.e., gender-combined).

As can be seen in FIGS. 4A-4G, the average and standard deviation changes were similar across the three populations (males, females, gender-combined) and were not significantly different for any of the segments. This is in contrast to separate stress and normal rest limit values which are different for males and females as was previously established in Slomka P. J., Nishina H., Berman D. S., et al., Automated quantification of myocardial perfusion SPECT using simplified normal limits, J. Nucl. Cardiol., 2005; 12(1): 66-77. Note that the change is not uniform across segments (or regions of the polar maps), which indicates the value of applying the normal change limit values for changes analysis. Based on these results, gender-combined normal change limit values were used in subsequent analysis.

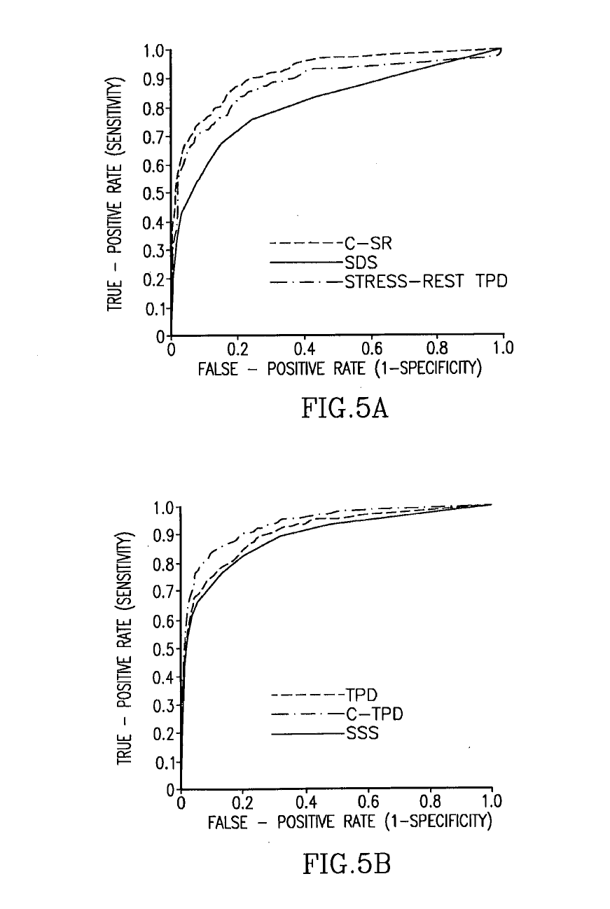

In FIGS. 5A and 5B, CAD was defined as greater than or equal to 70% stenosis by coronary angiography. The ROC curves in these figures were generated for the 997 test subjects.

FIG. 5A depicts ROC curves for the detection of CAD using the C-SR value (labeled “C-SR” in FIG. 5A), the difference between the stress TPD value and the rest TPD value (labeled “stress-rest TPD” in FIG. 5A), and the SDS value (labeled “SDS” in FIG. 5A). The AUC-ROC for the C-SR value, the difference between the stress TPD value and the rest TPD value, and the SDS value, were 0.92, 0.88, and 0.89 respectively (P<0.0001).

In FIG. 5B, the ROC curves for the detection of CAD using the C-TPD value (labeled “C-TPD” in FIG. 5B), the stress TPD value (labeled “TPD” in FIG. 5B), and the SSS value (labeled “SSS” in FIG. 5B) are displayed. Table 3 provides the AUC-ROC for the curves depicted in FIGS. 5A and 5B. As shown in Table 3 below, the AUC-ROC for the C-TPD value (which was 0.94) was significantly higher than the AUC-ROC for both the stress TPD value (which was 0.91) and the SSS value (which was 0.81).

TABLE 3

| Quantitative Variable | AUC-ROC |
|---|---|
| SSS | 0.89 |
| Stress-rest TPD | 0.88 |
| SDS | 0.81 |
| TPD | 0.91 |
| C-SR | 0.92 |
| C-TPD | 0.94 |

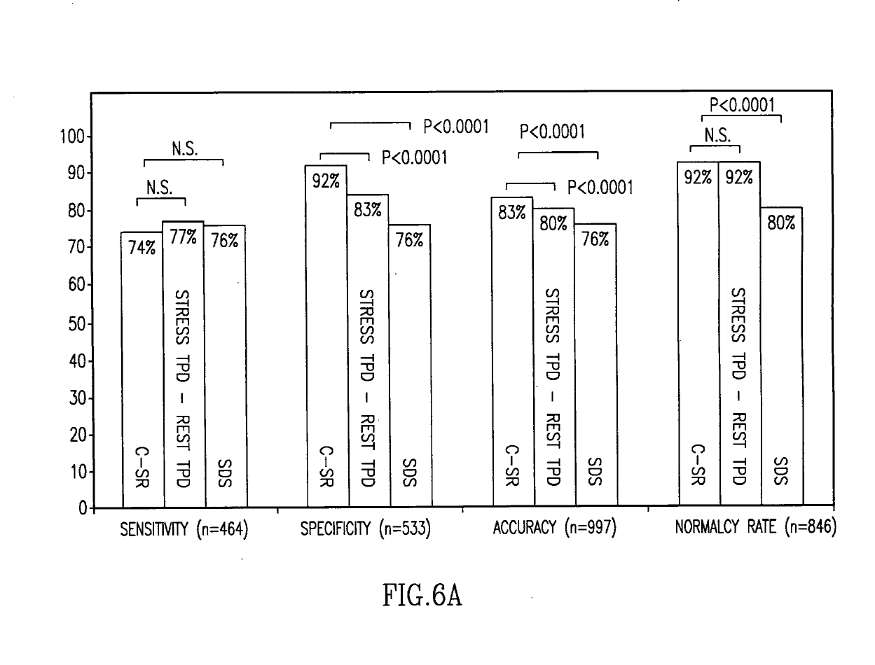

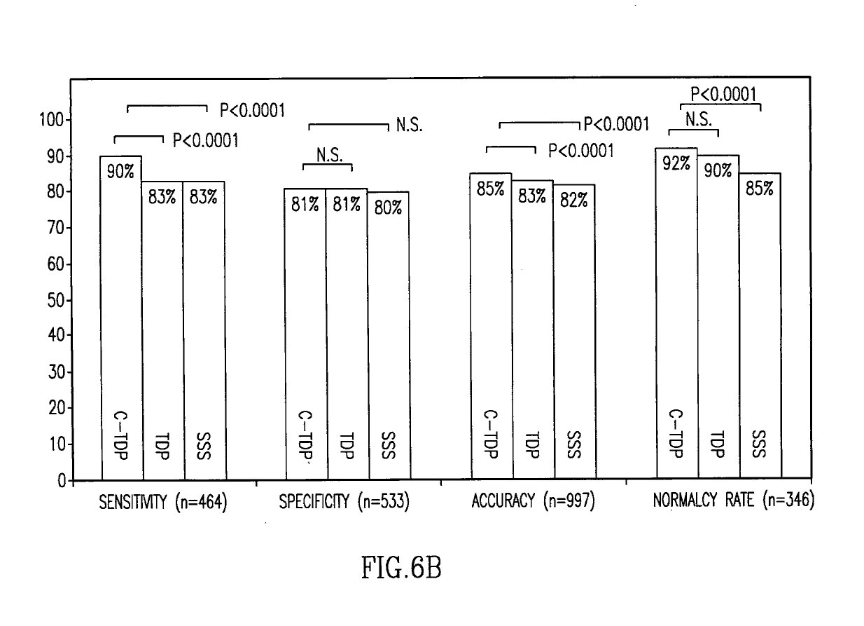

For comparison of sensitivity, specificity, and accuracy and normalcy rates, a cutoff of 3.0% was used for TPD variables, SSS>=3, SDS>=2 for automatic scores as previously established in Slomka P. J., Nishina H., Berman D. S., et al., Automated quantification of myocardial perfusion SPECT using simplified normal limits, J. Nucl. Cardiol., 2005; 12(1): 66-77. In general, sensitivity and accuracy of the C-TPD and C-SR values for detection of CAD was higher than standard measures of hypoperfusion. As can be seen in FIG. 6A, the C-SR value (labeled “C-SR” in FIG. 6A) had higher specificity and accuracy compared to that of the difference between the stress TPD value and the rest TPD value (labeled “stress TPD-rest TPD” in FIG. 6A) (P<0.0001), and the SDS value (labeled “SDS” in FIG. 6A) (P<0.0001). Sensitivity of the C-SR value was the same as the difference between the stress TPD value and the rest TPD value and the SDS value. As displayed in FIG. 6B, the specificity values, however, remained constant at 81% for the C-TPD value (labeled “C-TPD” in FIG. 6B) and the stress TPD value (labeled “TPD” in FIG. 6B). Accuracy for the C-TPD value was significantly higher than that of the stress TPD value (P=0.0045) and the SSS value (labeled “SSS” in FIG. 6B) (P<0.0001).

The angiographic group was considered separately, higher sensitivity and accuracy was also achieved using the C-TPD value compared to that of the stress TPD and SSS values (P<0.0001). Using the same cutoff, we found a higher sensitivity and accuracy using the C-TPD value compared to the stress TPD value (P<=0.0005). Specificity, however, was slightly lower for the C-TPD value (60%) compared to the stress TPD value (65%) (P<0.0001). Normalcy rate for the C-TPD value (92%) was similar to the normalcy rate of the stress TPD value (90%) (P=n.s.) and higher than the normalcy rate of the SSS value (85%). The C-SR value resulted in a normalcy rate of 92%, which was similar to the normalcy rate of the difference between the stress TPD value and the rest TPD value (92%) (P=n.s.) but higher than the normalcy rate of the SDS value (80%) (P<0.0001).

Ejection fraction is a fraction of blood pumped out of the left ventricle of the heart with each heart beat. For the angiographic group, the ejection fraction using stress was 59.4±12.3% and the ejection fraction using rest was 61.6±12.2%. For the LLK group (of normal subjects), the ejection fraction using stress was 67.6±8.2% and the ejection fraction using rest was 62.7+11.4%. As indicated in Table 4, the AUC-ROC values were higher (but not significantly) for vessels having greater than 50% lesion than for vessels having greater than 70% lesion.

TABLE 4

AUC-ROC

| Vessel | C-SR | C-TPD |
|---|---|---|
| LAD ≧ 70% lesion | 0.84 | 0.87 |
| LCX ≧ 70% lesion | 0.78 | 0.78 |
| RCA ≧ 70% lesion | 0.82 | 0.82 |
| LAD ≧ 50% lesion | 0.86 | 0.88 |
| LCX ≧ 50% lesion | 0.80 | 0.81 |
| RCA ≧ 50% lesion | 0.82 | 0.83 |

In summary, new quantitative MPS measures, the C-SR value and the C-TPD value, have been developed and validated. These new quantitative MPS measures are based on stress-rest changes and use normal change limit values for the purpose of CAD detection. Further, these new measures have been combined with traditional stress and rest quantification methods. The new measures can be derived in a fully automated manner and provide higher performance for detection of greater than or equal to 70% stenosis than any currently used quantitative measures such as the stress TPD value, the SSS value, and the SDS value.

Initially, separate sex-specific normal change limit values (for the change analysis) were derived from 40 male subjects and 40 female subjects to mirror the separate normal stress and rest limit values. However, the female and male normal change limit values were found to be the same for the stress-rest change. When gender-combined normal change limit values were tested, it was found that the two different approaches (separate sex-specific and gender-combined) resulted in the same AUC-ROC curve (which was about 0.94) for the C-TPD value. Therefore, in this study, the analyses were performed on the combined dataset (40 males and 40 females).

The C-TPD value, which combines stress-rest changes and stress hypoperfusion measures (e.g., on a pixel, sample, or coordinate basis) appears to be significantly better than standard change (the difference between the stress TPD value and the rest TPD value), the C-SR value, the stress TPD value, the SSS value, and the SDS value in predicting greater than 70% coronary artery stenosis. The C-TPD value yielded significant gains in the sensitivity, accuracy, and AUC-ROC over the other measures without compromising specificity. In addition, the C-TPD value improved the normalcy rate in patients with LLK of CAD.

The C-TPD value is determined by a method that uses a measure of change (or score) derived from normal change limit values when stress hypoperfusion is less apparent (using normal stress limit values). Referring to FIG. 7A-7E, accuracy is improved by replacing the subtle stress hypoperfusion value (determined using normal stress limit values) with change values (e.g., scores determined using normal change limit values). The MPS scan data used to generate FIGS. 7A-7E was collected from a 49 year old female patient with single vessel disease detected by coronary angiography (80% LAD coronary artery stenosis). However, her stress TPD value was 2.4% (depicted as black pixels on the patient stress polar map illustrated in FIG. 7C), while her C-SR value was 10% and C-TPD value was 11%. Thus, in this case, the C-SR and C-TPD values provided better accuracy than the stress TPD value.

However, in some circumstances, the change values alone may not be sufficient for predicting greater than 70% coronary artery stenosis in general due to the possibility of resting defects, for example in the case of resting ischemia or prior myocardial infarction. The C-TPD value (which combines measures using TPD together with change analysis) may provide improved detection of CAD over the C-SR value in such circumstances.

Using the C-SR and C-TPD values, gender differences should disappear because these approaches may sidestep attenuation issues. Because artifacts will usually be present on both stress and rest tomograms, in principle, these approaches may circumvent diaphragmatic attenuation issues.

Excluding patients with a coronary artery bypass graft (“CABG”) and prior-MI allows for the most stringent means of evaluating the accuracy of the detection of CAD by MPS. Including patients with known CAD tends to spuriously inflate sensitivity. Comparisons of different methods within a given institution or between institutions are more valid with these exclusions as the patient populations which includes known CAD often vary in their number as well as their severity.

A potentially limiting factor of the proposed technique is that it may not be used when the rest scan is unavailable or is of an unacceptable quality. In addition, assessment of the severity of the stenosis on angiograms has its own limitations in determining the physiologically significant lesions. See White C. W., Wright C. B., Doty D. B. et al., Does visual interpretation of the coronary arteriogram predict the physiologic importance of a coronary stenosis?, N. Engl. J. Med., 1984; 310:819-824. In the study described above, patients with prior myocardial infarction or revascularization were excluded because the presence of a severe perfusion defect associated with a myocardial infarct might artificially elevate the sensitivity for detection of CAD in a population where the question of disease detection is not relevant. In the study, established cut-off values were used to compare a clinically realistic operating point based on balancing the sensitivity with specificity. See Wolak A., Slomka P. J., Fish M. B., Lorenzo S., Berman D. S., Germano G., Quantitative diagnostic performance of myocardial perfusion SPECT with attenuation correction in women, J. Nucl. Med., 2008; 49(6):915-22. In clinical practice, a specific threshold may be applied to classify patients. In the study, the performance of the new software in that role was evaluated.

The C-SR value and the C-TPD value are each a novel and improved measure for quantification ischemia by MPS that use normal limit values of stress-rest change (e.g., the normal change limit values) for the detection of CAD. The analysis of the performance of these measures in comparison with standard methods indicates this new approach provides improved CAD detection as studied in a large group of patients as compared to current quantitative approaches.

### Computing Device

FIG. 8 is a diagram of hardware and an operating environment in conjunction with which implementations of the computing device 6 and/or the database 8 may be practiced. The description of FIG. 8 is intended to provide a brief, general description of suitable computer hardware and a suitable computing environment in which implementations may be practiced. Although not required, implementations are described in the general context of computer-executable instructions, such as program modules, being executed by a computer, such as a personal computer. Generally, program modules include routines, programs, objects, components, data structures, etc., that perform particular tasks or implement particular abstract data types.

Moreover, those skilled in the art will appreciate that implementations may be practiced with other computer system configurations, including hand-held devices, multiprocessor systems, microprocessor-based or programmable consumer electronics, network PCs, minicomputers, mainframe computers, and the like. Implementations may also be practiced in distributed computing environments where tasks are performed by remote processing devices that are linked through a communications network. In a distributed computing environment, program modules may be located in both local and remote memory storage devices.

The exemplary hardware and operating environment of FIG. 8 includes a general-purpose computing device in the form of a computing device 12. The computing device 6 and/or the database 8 may each be implemented using one or more computing devices like the computing device 12. Further, the computing device 6 and the database 8 may be implemented together on a single computing device like the computing device 12.

The computing device 12 includes a system memory 22, the processing unit 21, and a system bus 23 that operatively couples various system components, including the system memory 22, to the processing unit 21. There may be only one or there may be more than one processing unit 21, such that the processor of computing device 12 comprises a single central-processing unit (“CPU”), or a plurality of processing units, commonly referred to as a parallel processing environment. When multiple processing units are used, the processing units may be heterogeneous. By way of a non-limiting example, such a heterogeneous processing environment may include a conventional CPU, a conventional graphics processing unit (“GPU”), a floating-point unit (“FPU”), combinations thereof, and the like.

The computing device 12 may be a conventional computer, a distributed computer, or any other type of computer.

The system bus 23 may be any of several types of bus structures including a memory bus or memory controller, a peripheral bus, and a local bus using any of a variety of bus architectures. The system memory 112 (illustrated FIG. 3) may be substantially similar to the system memory 21. The system memory 21 may also be referred to as simply the memory, and includes read only memory (ROM) 24 and random access memory (RAM) 25. A basic input/output system (BIOS) 26, containing the basic routines that help to transfer information between elements within the computing device 12, such as during start-up, is stored in ROM 24. The computing device 12 further includes a hard disk drive 27 for reading from and writing to a hard disk, not shown, a magnetic disk drive 28 for reading from or writing to a removable magnetic disk 29, and an optical disk drive 30 for reading from or writing to a removable optical disk 31 such as a CD ROM, DVD, or other optical media.

The hard disk drive 27, magnetic disk drive 28, and optical disk drive 30 are connected to the system bus 23 by a hard disk drive interface 32, a magnetic disk drive interface 33, and an optical disk drive interface 34, respectively. The drives and their associated computer-readable media provide nonvolatile storage of computer-readable instructions, data structures, program modules, and other data for the computing device 12. It should be appreciated by those skilled in the art that any type of computer-readable media which can store data that is accessible by a computer, such as magnetic cassettes, flash memory cards, solid state memory devices (“SSD”), USB drives, digital video disks, Bernoulli cartridges, random access memories (RAMs), read only memories (ROMs), and the like, may be used in the exemplary operating environment. As is apparent to those of ordinary skill in the art, the hard disk drive 27 and other forms of computer-readable media (e.g., the removable magnetic disk 29, the removable optical disk 31, flash memory cards, SSD, USB drives, and the like) accessible by the processing unit 21 may be considered components of the system memory 22.

A number of program modules may be stored on the hard disk drive 27, magnetic disk 29, optical disk 31, ROM 24, or RAM 25, including an operating system 35, one or more application programs 36, other program modules 37, and program data 38. A user may enter commands and information into the computing device 12 through input devices such as a keyboard 40 and pointing device 42. Other input devices (not shown) may include a microphone, joystick, game pad, satellite dish, scanner, touch sensitive devices (e.g., a stylus or touch pad), video camera, depth camera, or the like. These and other input devices are often connected to the processing unit 21 through a serial port interface 46 that is coupled to the system bus 23, but may be connected by other interfaces, such as a parallel port, game port, a universal serial bus (USB), or a wireless interface (e.g., a Bluetooth interface). A monitor 47 or other type of display device is also connected to the system bus 23 via an interface, such as a video adapter 48. In addition to the monitor, computers typically include other peripheral output devices (not shown), such as speakers, printers, and haptic devices that provide tactile and/or other types physical feedback (e.g., a force feed back game controller).

The monitor 47 may be used to display a three or two dimensional representation of the left ventricle. By way of a non-limiting example, referring to FIG. 1, the monitor 47 (see FIG. 8) may display a visual representation of the normal subject stress scan data 134, normal subject rest scan data 136, normal subject change polar maps 138, normal change limit polar map 139, normal stress limit map 9S, and/or normal rest limit map 9R. Further, referring to FIG. 3, the monitor 47 (see FIG. 8) may display a visual representation of the patient stress scan data 114, patient rest scan data 116, patient stress polar map 124, patient rest polar map 126, patient change polar map 142, patient C-SR value 158, and/or patient C-TPD value 172.

The input devices described above are operable to receive user input and selections. Together the input and display devices may be described as providing a user interface. The input devices may be used to indicate whether to calculate the C-TPD value in decision block 170 of the method 100 illustrated in FIG. 2. The input devices may be used to direct the computing device 6 (see FIG. 1) to perform the optional block 155 of the method 100 illustrated in FIG. 2. The input devices may also be used to direct the computing device 6 (see FIG. 1) to perform the optional block 160 of the method 100 illustrated in FIG. 2. Further, the input devices may be used to enter and/or modify the C-SR threshold value 162, SR/TPD threshold value 192, and/or the C-TPD threshold value 194. The user interface may be used by the computing device 12 when executing the change analysis module 128 to indicate to a user that a patient has stenosis, hypoperfusion, ischemia, CAD, a combination thereof, and the like.

The computing device 12 may operate in a networked environment using logical connections to one or more remote computers, such as remote computer 49. These logical connections are achieved by a communication device coupled to or a part of the computing device 12 (as the local computer). Implementations are not limited to a particular type of communications device. The remote computer 49 may be another computer, a server, a router, a network PC, a client, a memory storage device, a peer device or other common network node, and typically includes many or all of the elements described above relative to the computing device 12. The remote computer 49 may be connected to a memory storage device 50. The logical connections depicted in FIG. 8 include a local-area network (LAN) 51 and a wide-area network (WAN) 52. Such networking environments are commonplace in offices, enterprise-wide computer networks, intranets and the Internet.

Those of ordinary skill in the art will appreciate that a LAN may be connected to a WAN via a modem using a carrier signal over a telephone network, cable network, cellular network, or power lines. Such a modem may be connected to the computing device 12 by a network interface (e.g., a serial or other type of port). Further, many laptop computers may connect to a network via a cellular data modem.

When used in a LAN-networking environment, the computing device 12 is connected to the local area network 51 through a network interface or adapter 53, which is one type of communications device. When used in a WAN-networking environment, the computing device 12 typically includes a modem 54, a type of communications device, or any other type of communications device for establishing communications over the wide area network 52, such as the Internet. The modem 54, which may be internal or external, is connected to the system bus 23 via the serial port interface 46. In a networked environment, program modules depicted relative to the personal computing device 12, or portions thereof, may be stored in the remote computer 49 and/or the remote memory storage device 50. It is appreciated that the network connections shown are exemplary and other means of and communications devices for establishing a communications link between the computers may be used.

The computing device 12 and related components have been presented herein by way of particular example and also by abstraction in order to facilitate a high-level view of the concepts disclosed. The actual technical design and implementation may vary based on particular implementation while maintaining the overall nature of the concepts disclosed.

When executed by one or more processors (e.g., the processing unit 21), the image processing module 118 may cause the one or more processors to perform block 120 of the method 100. Further, the system memory 112 may store instructions that when executed by one or more processors, instruct the scanning device 5 to perform an MPS scan. Further, the system memory 112 may store instructions that when executed by one or more processors, analyze the signal transmitted by the ECG 10 to identify points in the cardiac cycle and after identifying one or more points, instruct the scanning device 5 to perform an MPS scan.

The system memory 112 may store instructions for determining the normal stress limit values and/or the normal rest limit values. Further, the system memory 112 may store instructions for constructing the normal stress limit polar map 9S and storing the normal stress limit polar map in the database 8. The system memory 112 may also store instructions for constructing the normal rest limit polar map 9R and storing the normal rest limit polar map in the database 8.

When executed by one or more processors (e.g., the processing unit 21), the normal change limits module 127 may cause the one or more processors to perform block 130 of the method 100. When executed by one or more processors (e.g., the processing unit 21), the change analysis module 128 may cause the one or more processors to perform blocks 140-190 of the method 100.

Any of the instructions described above, including the instructions of each of the modules 118, 127, and 128, may be stored on one or more non-transitory computer-readable media. The instructions described above are executable by one or more processors (e.g., the processing unit 21) and when executed perform the functions described above.

The foregoing described embodiments depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

The invention claimed is:

1. A computer implemented method for use with a patient having a heart, the method comprising:
obtaining patient stress scan data;
obtaining patient rest scan data;
determining patient stress-rest change values based on the patient stress scan data and the patient rest scan data;
obtaining normal change limit values; and
determining whether the patient has ischemia by comparing the patient stress-rest change values with the normal change limit values.

2. The method of claim 1, further comprising:
generating a patient stress polar map from the patient stress scan data, the patient stress polar map comprising stress count values;
obtaining normal stress limit values;
assigning a score within a predetermined range to each of the patient stress count values in the patient stress polar map based on the normal stress limit values;
generating a patient change polar map from the patient stress-rest change values, the patient change polar map comprising a plurality of change values, each change value corresponding to a stress count value in the patient stress polar map,
wherein comparing the patient stress-rest change values and the normal change limit values comprises:
(a) assigning a score within the predetermined range to each of the patient stress-rest change values in the patient change polar map based on the normal change limit values; and
(b) comparing each score assigned to the patient stress count values in the patient stress polar map to a first threshold value, and if the score is less than the first threshold value, replacing the score assigned to the patient stress count values in the patient stress polar map with the score assigned to the change value in the patient change polar map that corresponds to the stress count value, and averaging the scores and replacement scores assigned to the stress count values in the patient stress polar map; and
determining whether the patient has ischemia comprises comparing the average score to a second threshold value.

3. The method of claim 1, further comprising:
generating a patient stress polar map from the patient stress scan data, the patient stress polar map comprising stress count values;
obtaining normal stress limit values;
assigning a score within a predetermined range having a maximum score to each of the patient stress count values in the patient stress polar map based on the normal stress limit values;
generating a patient change polar map from the patient stress-rest change values, the patient change polar map comprising a plurality of change values, each of the plurality of change values corresponding to a stress count value in the patient stress polar map,
wherein comparing the patient stress-rest change values and the normal change limit values comprises:
(a) assigning a score within the predetermined range to each of the patient stress-rest change values in the patient change polar map based on the normal change limit values; and
(b) comparing each score assigned to the patient stress count values in the patient stress polar map to a first threshold value, and if the score is less than the first threshold value, replacing the score assigned to the patient stress count values in the patient stress polar map with the score assigned to the change value in the patient change polar map that corresponds to the stress count value, averaging the scores and replacement scores assigned to the stress count values in the patient stress polar map to obtain an average score, and dividing the average score by the maximum score to obtain a percentage value; and
determining whether the patient has ischemia comprises comparing the percentage value to a second threshold value.

4. The method of claim 1, wherein determining the patient stress-rest change values further comprises:
generating a patient stress polar map from the patient stress scan data, the patient stress polar map comprising stress count values;
generating a patient rest polar map from the patient rest scan data, the patient rest polar map comprising rest count values;
co-registering the patient stress polar map and the patient rest polar map such that each stress count value of the patient stress polar map corresponds to a rest count value of the patient rest polar map;
normalizing the stress count values and the rest count values of the co-registered patient stress polar map and the patient rest polar map; and
subtracting the normalized stress count values of the patient stress polar map from the normalized rest count values of the patient rest polar map.

5. The method of claim 1, wherein comparing the patient stress-rest change values and the normal change limit values further comprises:
(a) assigning a score to each of the patient stress-rest change values based on the normal change limit values; and
(b) averaging the scores assigned to the patient stress-rest change values to obtain an average score; and
determining whether the patient has ischemia comprises comparing the average score to a threshold value.

6. The method of claim 1, wherein comparing the patient stress-rest change values and the normal change limit values further comprises:
(a) assigning a score within a range comprising a maximum score to each of the patient stress-rest change values based on the normal change limit values; and
(b) averaging the scores assigned to the patient stress-rest change values to obtain an average score; and
(c) dividing the average score by the maximum score to obtain a change percentage; and
determining whether the patient has ischemia comprises comparing the change percentage to a threshold value.

7. The method of claim 1, wherein obtaining the patient stress scan data further comprises performing a stress myocardial perfusion single-photon emission computerized tomography (“MPS”) scan on the patient when the patient's heart is operating under stress; and
obtaining the patient rest scan data further comprises performing a rest MPS scan on the patient when the patient's heart is operating at rest.

8. The method of claim 1 for use with a database storing the normal change limit values, wherein obtaining the normal change limit values comprises:
retrieving the normal change limit values from the database.

9. The method of claim 1 for use with a plurality of normal subjects, wherein obtaining the normal change limit values comprises:
for each of the plurality of normal subjects, obtaining subject stress scan data;
for each of the plurality of normal subjects, obtaining subject rest scan data;
for each of the plurality of normal subjects, obtaining subject stress-rest change values based on the subject stress scan data and the subject rest scan data; and
determining the normal change limit values based on the subject stress-rest change values obtained for the plurality of normal subjects.

10. The method of claim 1 for use with a plurality of normal subjects, wherein obtaining the normal change limit values comprises:
(a) generating a plurality of co-registered subject change polar maps for the plurality of normal subjects, and generating the plurality of co-registered subject change polar maps comprising for each of the plurality of normal subjects:
obtaining subject stress scan data;
obtaining subject rest scan data;
generating a subject stress polar map from the subject stress scan data, the subject stress polar map comprising stress count values;
generating a subject rest polar map from the patient rest scan data, the subject rest polar map comprising rest count values;
co-registering the subject stress polar map and the subject rest polar map such that each stress count value of the subject stress polar map corresponds to a rest count value of the subject rest polar map;
normalizing the stress count values and the rest count values of the co-registered subject stress polar map and the subject rest polar map; and
generating a subject change polar map by subtracting the normalized stress count values of the subject stress polar map from the normalized rest count values of the subject rest polar map, the subject change polar map comprising a plurality of polar coordinates, each polar coordinate being associated with a subject change value; and
(b) for each coordinate in the plurality of co-registered subject change polar maps, calculating a change limit value based on the subject change values associated with the coordinate in each of the plurality of co-registered subject change polar maps.

11. The method of claim 10, wherein for each coordinate in the plurality of co-registered subject change polar maps, calculating the change limit value based on the subject change values associated with the coordinate in each of the plurality of co-registered subject change polar maps comprises:
calculating a mean and standard deviation of the subject change values associated with the coordinate in each of the plurality of co-registered subject change polar maps, the change limit value being equal to a sum of the mean and twice the standard deviation.

12. The method of claim 10 for use with a database storing the subject stress scan data for each of the plurality of normal subjects and the subject rest scan data for each of the plurality of normal subjects, wherein obtaining the subject stress scan data for each of the plurality of normal subjects comprises retrieving the subject stress scan data from the database; and
obtaining the subject rest scan data for each of the plurality of normal subjects comprises retrieving the subject rest scan data from the database.

13. The method of claim 10 for use with the plurality of normal subjects each comprising a heart, wherein obtaining wherein obtaining the subject stress scan data for each of the plurality of normal subjects comprises performing a stress MPS scan on the subject when the subject's heart is operating under stress; and
obtaining the subject rest scan data for each of the plurality of normal subjects comprises performing a rest MPS scan on the subject when the subject's heart is operating at rest.

14. A computer implemented method for use with a patient stress polar map comprising stress count values, a patient rest polar map comprising rest count values, and a normal change limit polar map comprising change limit values, the patient stress polar map and the patient rest polar map being co-registered with one another, the normal change limit polar map being co-registered with both the patient stress polar map and the patient rest polar map, the method comprising:
creating a patient change polar map by subtracting the rest count values of the patient rest polar map from the stress count values of the co-registered patient stress polar map, the patient change polar map being co-registered with the normal change limit polar map;
comparing the patient change polar map with the normal change limit polar map to detect one or more regions in the patient change polar map in which the change value in the patient change polar map is greater than the change limit value in the co-registered normal change limit polar map; and
identifying the one or more regions detected as having perfusion abnormalities.

15. The method of claim 14, wherein identifying the one or more regions detected as having perfusion abnormalities comprises displaying the one or more regions.

16. The method of claim 14, wherein the patient change polar map comprises a plurality of patient change values, and comparing the patient change polar map with the normal change limit polar map comprises:
assigning a score to each of the plurality of patient change values based at least in part on whether the patient change value is greater than the normal change limit value with which the patient change value is co-registered, the score being within a predetermined range comprising a maximum score;
averaging the scores assigned to the patient stress values in the patient stress polar map to obtain an average score, and dividing the average score by the maximum score to obtain a percentage value; and
detecting the entire patient stress polar map indicates a significant perfusion deficiency by comparing the percentage value to a threshold percentage value.

17. The method of claim 14, wherein the threshold percentage value is at least 5% and less than 10% and the entire patient stress polar map indicates a significant perfusion deficiency when the percentage value is greater than the threshold percentage value.

18. A system for use with a patient comprising a heart, patient stress scan data obtained for the patient when the patient's heart was operating under stress, and patient rest scan data obtained for the patient when the patient's heart was operating at rest, the system comprising:
a data storage device storing a normal change limit polar map comprising change limit values; and
a computing device connected to the data storage device, the computing device being configured to:
generate a patient stress polar map from the patient stress scan data, the patient stress polar map comprising stress count values,
generate a patient rest polar map from the patient rest scan data, the patient rest polar map comprising rest count values,
co-register the patient stress polar map and the patient rest polar map,
co-register both the patient stress polar map and the patient rest polar map with the normal change limit polar map,
creating a patient change polar map by subtracting the rest count values of the patient rest polar map from the stress count values of the co-registered patient stress polar map, the patient change polar map being co-registered with the normal change limit polar map,
compare the patient change polar map with the normal change limit polar map to detect one or more regions in the patient change polar map in which the change value in the patient change polar map is greater than the change limit value in the co-registered normal change limit polar map, and
identify the one or more regions detected as having perfusion abnormalities.

19. The system of claim 18 for use with a user, wherein the computing device comprises a display device configured to display the one or more regions detected as having perfusion abnormalities to the user.

20. The system of claim 18, wherein the patient change polar map comprises a plurality of patient change values, and comparing the patient change polar map with the normal change limit polar map comprises:
assigning a score to each of the plurality of patient change values based at least in part on whether the patient change value is greater than the normal change limit value with which the patient change value is co-registered, the score being within a predetermined range comprising a maximum score;
averaging the scores assigned to the patient stress values in the patient stress polar map to obtain an average score, and dividing the average score by the maximum score to obtain a percentage value; and
detecting the entire patient stress polar map indicates a significant perfusion deficiency by comparing the percentage value to a threshold percentage value.

21. The system of claim 18, wherein the threshold percentage value is at least 5% and less than 10%, and the computing device detects the entire patient stress polar map indicates a significant perfusion deficiency when the percentage value is greater than the threshold percentage value.

22. A system for use with a patient comprising a heart, the system comprising:
a scanning device configured to scan the patient's heart when the patient's heart is operating under stress to obtain patient stress scan data and to scan the patient's heart when the patient's heart is operating at rest to obtain patient rest scan data;
a data storage device storing normal change limit values; and
a computing device connected to the data storage device and the scanner device, the computing device being configured to obtain the patient stress scan data and the patient rest scan data from the scanner device, determine patient stress-rest change values based on the patient stress scan data and the patient rest scan data, obtain the normal change limit values from the data storage device, and determine whether the patient has ischemia by comparing the patient stress-rest change values with the normal change limit values.

23. The system of claim 22, wherein the computing device comprises a display device configured to display a result of the determination of whether the patient has ischemia.

24. The system of claim 22, wherein the scanning device is a single-photon emission computerized tomography (“SPECT”) scanner.

25. The system of claim 22, wherein the patient stress scan data and the patient rest scan data comprise images of a left ventricle of the patient's heart.

26. The system of claim 22, further comprising an electrocardiogram configured to detect a cardiac cycle of the patient's heart, and transmit a signal to the computing device based on the cardiac cycle detected, the computing device being further configured to:
analyze the signal to identify a point in the cardiac cycle; and
after detecting the point in the cardiac cycle, instruct the scanning device to collect the patient stress scan data and the patient rest scan data.

27. The system of claim 22, wherein the data storage device stores normal stress limit values, and the computing device is further configured to:
generate a patient stress polar map from the patient stress scan data, the patient stress polar map comprising stress count values;
assign a score within a predetermined range to each of the patient stress count values in the patient stress polar map based on the normal stress limit values;
generate a patient change polar map from the patient stress-rest change values, the patient change polar map comprising a plurality of change values, each change value corresponding to a stress count value in the patient stress polar map,
wherein comparing the patient stress-rest change values and the normal change limit values comprises:
(a) assigning a score within the predetermined range to each of the patient stress-rest change values in the patient change polar map based on the normal change limit values; and
(b) comparing each score assigned to the patient stress count values in the patient stress polar map to a first threshold value, and if the score is less than the first threshold value, replacing the score assigned to the patient stress count values in the patient stress polar map with the score assigned to the change value in the patient change polar map that corresponds to the stress count value, and averaging the scores and replacement scores assigned to the stress count values in the patient stress polar map to obtain an average score; and
determine whether the patient has ischemia by comparing the average score to a second threshold value.

28. The system of claim 22, wherein comparing the patient stress-rest change values and the normal change limit values further comprises:
(a) assigning a score to each of the patient stress-rest change values based on the normal change limit values; and
(b) averaging the scores assigned to the patient stress-rest change values to obtain an average score; and
the computing device is configured to determine whether the patient has ischemia by comparing the average score to a threshold value.

29. One or more computer readable media comprising instructions executable by one or more processors and when executed by the one or more processors causing the one or more processors to perform a method comprising:
obtaining patient stress scan data;
obtaining patient rest scan data;
determining patient stress-rest change values based on the patient stress scan data and the patient rest scan data;
obtaining normal change limit values; and
determining whether the patient has ischemia by comparing the patient stress-rest change values with the normal change limit values.

30. The one or more computer readable media of claim 29, further comprising normal stress limit values, wherein the method further comprises:
generating a patient stress polar map from the patient stress scan data, the patient stress polar map comprising stress count values;
assigning a score within a predetermined range to each of the patient stress count values in the patient stress polar map based on the normal stress limit values;
generating a patient change polar map from the patient stress-rest change values, the patient change polar map comprising a plurality of change values, each change value corresponding to a stress count value in the patient stress polar map,
wherein comparing the patient stress-rest change values and the normal change limit values comprises:
(a) assigning a score within the predetermined range to each of the patient stress-rest change values in the patient change polar map based on the normal change limit values; and
(b) comparing each score assigned to the patient stress count values in the patient stress polar map to a first threshold value, and if the score is less than the first threshold value, replacing the score assigned to the patient stress count values in the patient stress polar map with the score assigned to the change value in the patient change polar map that corresponds to the stress count value, and averaging the scores and replacement scores assigned to the stress count values in the patient stress polar map; and
determining whether the patient has ischemia comprises comparing the average score to a second threshold value.

31. The one or more computer readable media of claim 29, wherein the method further comprises:
generating a patient stress polar map from the patient stress scan data, the patient stress polar map comprising stress count values;
generating a patient rest polar map from the patient rest scan data, the patient rest polar map comprising rest count values;
co-registering the patient stress polar map and the patient rest polar map such that each stress count value of the patient stress polar map corresponds to a rest count value of the patient rest polar map;
normalizing the stress count values and the rest count values of the co-registered patient stress polar map and the patient rest polar map; and
subtracting the normalized stress count values of the patient stress polar map from the normalized rest count values of the patient rest polar map.

32. The one or more computer readable media of claim 29, wherein the method further comprises:
(a) assigning a score to each of the patient stress-rest change values based on the normal change limit values; and
(b) averaging the scores assigned to the patient stress-rest change values to obtain an average score; and
determining whether the patient has ischemia comprises comparing the average score to a threshold value.

33. One or more computer readable media comprising normal change limit values and instructions executable by one or more processors and when executed by the one or more processors causing the one or more processors to perform a method comprising:
obtaining patient stress scan data;
obtaining patient rest scan data;
determining patient stress-rest change values based on the patient stress scan data and the patient rest scan data; and
determining whether the patient has ischemia by comparing the patient stress-rest change values with the normal change limit values.

34. One or more computer readable media comprising instructions executable by one or more processors and when executed by the one or more processors causing the one or more processors to perform a method comprising:
obtaining a patient stress polar map comprising stress count values;
obtaining a patient rest polar map comprising rest count values;
obtaining a normal change limit polar map comprising change limit values, the patient stress polar map and the patient rest polar map being co-registered with one another, the normal change limit polar map being co-registered with both the patient stress polar map and the patient rest polar map;
determining a patient change polar map by subtracting the rest count values of the patient rest polar map from the stress count values of the co-registered patient stress polar map, the patient change polar map being co-registered with the normal change limit polar map;
comparing the patient change polar map with the normal change limit polar map to detect one or more regions in the patient change polar map in which the change value in the patient change polar map is greater than the change limit value in the co-registered normal change limit polar map; and
identifying the one or more regions detected as having perfusion abnormalities.

35. The one or more computer readable media of claim 34, wherein the method further comprises: displaying the one or more regions detected as having perfusion abnormalities.

36. The one or more computer readable media of claim 34, wherein the patient change polar map comprises a plurality of patient change values, and comparing the patient change polar map with the normal change limit polar map comprises:
assigning a score to each of the plurality of patient change values based at least in part on whether the patient change value is greater than the normal change limit value with which the patient change value is co-registered, the score being within a predetermined range comprising a maximum score;
averaging the scores assigned to the patient stress values in the patient stress polar map to obtain an average score, and dividing the average score by the maximum score to obtain a percentage value; and
detecting the entire patient stress polar map indicates a significant perfusion deficiency by comparing the percentage value to a threshold percentage value.

37. The one or more computer readable media of claim 34, wherein the threshold percentage value is at least 5% and less than 10%, and the entire patient stress polar map indicates a significant perfusion deficiency when the percentage value is greater than the threshold percentage value.

38. A computer implemented method for use with a patient stress polar map comprising stress count values, a patient rest polar map comprising rest count values, a normal stress limit polar map comprising stress limit values, and a normal change limit polar map comprising change limit values, the patient stress polar map and the patient rest polar map being co-registered with one another, the normal change limit polar map being co-registered with both the patient stress polar map and the patient rest polar map, the normal stress limit polar map being co-registered with the patient stress polar map, the method comprising:
creating a patient change polar map by subtracting the rest count values of the patient rest polar map from the stress count values of the co-registered patient stress polar map, the patient change polar map comprising patient change values and being co-registered with the normal change limit polar map;
comparing the patient stress polar map with the normal stress limit polar map and assigning a score to each of the patient stress values of the patient stress polar map based on the comparison, the score being a value within a predetermined range;
for each patient stress value assigned a score below a predetermined threshold value, identifying a patient change value in the patient change polar map co-registered with the patient stress value, identifying a normal change limit value in the normal change limit polar map co-registered with the identified patient change value, determining a score for the patient change value based on the identified normal change limit value and the identified patient change value, the score being a value within the predetermined range, and assigning the score to the patient stress value in the patient stress polar map; and
determining whether the patient stress polar map indicates a significant perfusion deficiency based on the scores assigned to the patient stress values in the patient stress polar map.

39. The method of claim 38, wherein the predetermined range comprises a maximum score, and determining whether the patient stress polar map indicates a significant perfusion deficiency comprises:
averaging the scores assigned to the patient stress values in the patient stress polar map to obtain an average score, and dividing the average score by the maximum score to obtain a percentage value; and
determining the patient stress polar map indicates a significant perfusion deficiency by comparing the percentage value to a threshold percentage value.

40. The method of claim 39, wherein the predetermined range comprises a maximum score, and determining whether the patient stress polar map indicates a significant perfusion deficiency comprises:
averaging the scores assigned to the patient stress values in the patient stress polar map to obtain an average score; and
determining the patient stress polar map indicates a significant perfusion deficiency by comparing the average score to a threshold percentage value.
